(19) United States
(12) Patent Application Publication
(10) Pub. No.: US 2024/0041870 A1
(43) Pub. Date: Feb. 8, 2024

(54) METHODS OF TREATING SYSTEMIC LUPUS ERYTHEMATOSUS

(71) Applicant: AbbVie Inc., North Chicago, IL (US)

(72) Inventors: Alan W. Friedman, Libertyville, IL (US); Mohamed-Eslam F. Mohamed, Gurnee, IL (US)

(21) Appl. No.: 18/360,151

(22) Filed: Jul. 27, 2023

Related U.S. Application Data
(60) Provisional application No. 63/393,158, filed on Jul. 28, 2022.
(60) Provisional application No. 63/411,785, filed on Sep. 30, 2022.

(51) Int. Cl. A61K 31/4985; A61K 31/473; A61K 31/573; A61K 31/42; A61P 37/00

(52) U.S. Cl. CPC A61K 31/4985; A61K 31/473; A61K 31/573; A61K 31/42; A61P 37/00

(57) ABSTRACT

The present disclosure is directed to methods for treating systemic lupus erythematosus (SLE) using the selective JAK1 inhibitor upadacitinib.

### CROSS-REFERENCE TO RELATED APPLICATIONS

This application claims the benefit of U.S. Provisional Application No. 63/393,158, filed Jul. 28, 2022, and claims the benefit of U.S. Provisional Application No. 63/411,785, filed Sep. 30, 2022, each of which are herein incorporated by reference in their entirety.

### FIELD OF THE DISCLOSURE

The present disclosure is directed to methods of treating systemic lupus erythematosus (SLE) with the selective JAK1 inhibitor, upadacitinib.

### BACKGROUND OF THE DISCLOSURE

Systemic lupus erythematosus (SLE) is an autoimmune disease characterized by antibodies to nuclear and cytoplasmic antigens, multisystem inflammation, protean clinical manifestations, and a relapsing and remitting course. SLE causes widespread inflammation and tissue damage in the affected organs, which may include joints, skin, brain, lungs, kidneys, and blood vessels. Symptoms of SLE vary depending on the affected organs, but may include fatigue, skin rashes, fevers, and pain or swelling in the joints.

Currently, there is no cure for SLE, and existing therapies focus on improving quality of life by controlling symptoms and minimizing flare-ups, primarily through use of immunosuppressive drugs such as hydroxychloroquine and corticosteroids, and immunomodulators such as belimumab and anifrolumab. Despite these treatments, significant unmet therapeutic needs remain for SLE patients. Provided herein are methods of treating SLE.

### SUMMARY OF THE DISCLOSURE

The present disclosure provides methods for treating systemic lupus erythematosus (SLE) with the selective JAK1 inhibitor, upadacitinib. The treatment methods generally comprise administering to a patient in need thereof a therapeutically effective amount of upadacitinib.

In one aspect is provided a method of treating a human patient having moderately to severely active systemic lupus erythematosus, the method comprising orally administering once daily to the patient 30 mg of upadacitinib.

In another aspect is provided a method of treating a human patient having moderately to severely active systemic lupus erythematosus, the method comprising orally administering once daily to the patient 15 mg of upadacitinib.

In yet another aspect is provided a method of treating a human patient having moderately to severely active systemic lupus erythematosus, the method comprising orally administering once daily to the patient 45 mg of upadacitinib. In some embodiments, the 45 mg of upadacitinib is an induction dose, followed by orally administering once daily to the patient a maintenance dose. In some embodiments, the maintenance dose is 30 mg. In some embodiments, the maintenance dose is 15 mg.

In some embodiments, the method results in a SLE Responder Index (SRI)-4 response and Prednisone Equivalent Steroid Dose ≤10 mg at 52 weeks after the first daily administration.

In some embodiments, the method results in a SLE Responder Index (SRI)-4 response at 52 weeks after the first daily administration.

In some embodiments, the method results in a BILAG-Based Combined Lupus Assessment (BICLA) response at 52 weeks after the first daily administration.

In some embodiments, the method results in a Lupus Low Disease Activity State (LLDAS) response at 52 weeks after the first daily administration.

In some embodiments, the method results in a reduction in steroid burden, assessed as change from Baseline in prednisone equivalent steroid dose at Week 52.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 52.

In some embodiments, the method results in a SLE Responder Index (SRI)-4 response and Prednisone Equivalent Steroid Dose 10 mg at 48 weeks after the first daily administration.

In some embodiments, the method results in a SLE Responder Index (SRI)-4 response at 48 weeks after the first daily administration.

In some embodiments, the method results in a BILAG-Based Combined Lupus Assessment (BICLA) response at 48 weeks after the first daily administration.

In some embodiments, the method results in a Lupus Low Disease Activity State (LLDAS) response at 48 weeks after the first daily administration.

In some embodiments, the method results in a reduction in steroid burden, assessed as change from Baseline in prednisone equivalent steroid dose at Week 48.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 48.

In some embodiments, the method results in a SLE Responder Index (SRI)-4 response and Prednisone Equivalent Steroid Dose ≤10 mg at 24 weeks after the first daily administration.

In some embodiments, the method results in a SLE Responder Index (SRI)-4 response at 24 weeks after the first daily administration.

In some embodiments, the method results in a BILAG-Based Combined Lupus Assessment (BICLA) response at 24 weeks after the first daily administration.

In some embodiments, the method results in a Lupus Low Disease Activity State (LLDAS) response at 24 weeks after the first daily administration.

In some embodiments, the method results in a reduction in steroid burden, assessed as change from Baseline in prednisone equivalent steroid dose at Week 24.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 24.

In some embodiments, the method results in a SLE Responder Index (SRI)-4 response and Prednisone Equivalent Steroid Dose 10 mg at 20 weeks after the first daily administration.

In some embodiments, the method results in a SLE Responder Index (SRI)-4 response at 20 weeks after the first daily administration.

In some embodiments, the method results in a BILAG-Based Combined Lupus Assessment (BICLA) response at 20 weeks after the first daily administration.

In some embodiments, the method results in a Lupus Low Disease Activity State (LLDAS) response at 20 weeks after the first daily administration.

In some embodiments, the method results in a reduction in steroid burden, assessed as change from Baseline in prednisone equivalent steroid dose at Week 20.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 20.

In some embodiments, the method results in a SLE Responder Index (SRI)-4 response and Prednisone Equivalent Steroid Dose 10 mg at 16 weeks after the first daily administration.

In some embodiments, the method results in a SLE Responder Index (SRI)-4 response at 16 weeks after the first daily administration.

In some embodiments, the method results in a BILAG-Based Combined Lupus Assessment (BICLA) response at 16 weeks after the first daily administration.

In some embodiments, the method results in a Lupus Low Disease Activity State (LLDAS) response at 16 weeks after the first daily administration.

In some embodiments, the method results in a reduction in steroid burden, assessed as change from Baseline in prednisone equivalent steroid dose at Week 16.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 16.

In some embodiments, the method results in a SLE Responder Index (SRI)-4 response and Prednisone Equivalent Steroid Dose ≤10 mg at 12 weeks after the first daily administration.

In some embodiments, the method results in a SLE Responder Index (SRI)-4 response at 12 weeks after the first daily administration.

In some embodiments, the method results in a BILAG-Based Combined Lupus Assessment (BICLA) response at 12 weeks after the first daily administration.

In some embodiments, the method results in a Lupus Low Disease Activity State (LLDAS) response at 12 weeks after the first daily administration.

In some embodiments, the method results in a reduction in steroid burden, assessed as change from Baseline in prednisone equivalent steroid dose at Week 12.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 12.

In some embodiments, the method results in a SLE Responder Index (SRI)-4 response and Prednisone Equivalent Steroid Dose 10 mg at 8 weeks after the first daily administration.

In some embodiments, the method results in a SLE Responder Index (SRI)-4 response at 8 weeks after the first daily administration.

In some embodiments, the method results in a BILAG-Based Combined Lupus Assessment (BICLA) response at 8 weeks after the first daily administration.

In some embodiments, the method results in a Lupus Low Disease Activity State (LLDAS) response at 8 weeks after the first daily administration.

In some embodiments, the method results in a reduction in steroid burden, assessed as change from Baseline in prednisone equivalent steroid dose at Week 8.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 8.

In some embodiments, the method results in a SLE Responder Index (SRI)-4 response and Prednisone Equivalent Steroid Dose ≤10 mg at 4 weeks after the first daily administration.

In some embodiments, the method results in a SLE Responder Index (SRI)-4 response at 4 weeks after the first daily administration.

In some embodiments, the method results in a BILAG-Based Combined Lupus Assessment (BICLA) response at 4 weeks after the first daily administration.

In some embodiments, the method results in a Lupus Low Disease Activity State (LLDAS) response at 4 weeks after the first daily administration.

In some embodiments, the method results in a reduction in steroid burden, assessed as change from Baseline in prednisone equivalent steroid dose at Week 4.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 4.

In some embodiments, the method results in a significant decrease in the levels of anti-dsDNA autoantibodies and complement C3 and C4.

In some embodiments, the method results in a significant increase in the numbers of circulating memory, naïve, and total B cells.

In some embodiments, the patient has had an inadequate response or intolerance to one or more disease-modifying antirheumatic drugs.

In some embodiments, the patient has had an inadequate response or intolerance to methotrexate.

In some embodiments, the patient has had an inadequate response or intolerance to an anti-malarial agent.

In some embodiments, the patient has had an inadequate response or intolerance to an immunomodulator.

In some embodiments, the patient has had an inadequate response or intolerance to a corticosteroid.

In some embodiments, the patient has had an inadequate response or intolerance to prednisone (or prednisone equivalent), an antimalarial agent, azathioprine, mycophenolate, leflunomide, cyclosporine, tacrolimus, or methotrexate.

In some embodiments, the patient has had an inadequate response or intolerance to belimumab.

In some embodiments, the patient has had an inadequate response or intolerance to rituximab.

In some embodiments, the patient has had an inadequate response or intolerance to anifrolumab.

In some embodiments, the patient is concomitantly treated with an anti-malarial agent.

In some embodiments, the patient is concomitantly treated with an immunomodulator.

In some embodiments, the patient is concomitantly treated with a corticosteroid.

In some embodiments, the patient is concomitantly treated with prednisone (or prednisone equivalent), an antimalarial agent, azathioprine, mycophenolate, leflunomide, cyclosporine, tacrolimus, or methotrexate.

### BRIEF DESCRIPTION OF THE DRAWINGS

FIG. 1 is a schematic illustration of a clinical study according to an embodiment of the disclosure.

FIG. 2 is a graphical depiction of response rate over time with respect to the primary endpoint (achievement of SRI-4 and Steroid Dose ≤10 mg prednisone equivalent QD) for subjects treated with placebo and upadacitinib 30 mg QD.

FIG. 3 is a graphical depiction of response rate over time with respect to achievement of SRI-4 for subjects treated with placebo and upadacitinib 30 mg QD.

FIG. 4 is a graphical depiction of response rate over time with respect to achievement of BICLA for subjects treated with placebo and upadacitinib 30 mg QD.

FIG. 5A is an exposure-response quartile plot for upadacitinib efficacy at week 24 for the efficacy endpoint of SLE Responder Index (SRI-4) and Steroid Dose ≤10 mg prednisone equivalent QD.

FIG. 5B is an exposure-response quartile plot for upadacitinib efficacy at week 24 for the efficacy endpoint of BILAG-Based Combined Lupus Assessment (BICLA).

FIG. 5C is an exposure-response quartile plot for upadacitinib efficacy at week 24 for the efficacy endpoint of SLE Responder Index (SRI-4).

FIG. 6 is a graphical depiction of response rate over 48 weeks with respect to the primary endpoint (achievement of SRI-4 and Steroid Dose ≤10 mg prednisone equivalent QD) for subjects treated with placebo and upadacitinib 30 mg QD.

FIG. 7 is a graphical depiction of response rate over 48 weeks with respect to achievement of SRI-4 for subjects treated with placebo and upadacitinib 30 mg QD.

FIG. 8 is a graphical depiction of response rate over 48 weeks with respect to achievement of BICLA for subjects treated with placebo and upadacitinib 30 mg QD.

FIG. 9 is a graphical depiction of time to first flare over 48 weeks for subjects treated with placebo and upadacitinib 30 mg QD.

FIG. 10A is an exposure-response quartile plot for upadacitinib efficacy at week 48 for the efficacy endpoint of SLE Responder Index (SRI-4) and Steroid Dose ≤10 mg prednisone equivalent QD.

FIG. 10B is an exposure-response quartile plot for upadacitinib efficacy at week 48 for the efficacy endpoint of BTLAG-Based Combined Lupus Assessment (BICLA).

FIG. 10C is an exposure-response quartile plot for upadacitinib efficacy at week 48 for the efficacy endpoint of SLE Responder Index (SRI-4).

### DETAILED DESCRIPTION OF THE DISCLOSURE

This written description uses examples to disclose the invention and also to enable any person skilled in the art to practice the invention, including making and using any of the disclosed compositions, and performing any of the disclosed methods or processes. The patentable scope of the invention is defined by the claims, and may include other examples that occur to those skilled in the art. Such other examples are intended to be within the scope of the claims if they have elements that do not differ from the literal language of the claims, or if they include equivalent elements.

### I. Definitions

Section headings as used in this section and the entire disclosure are not intended to be limiting.

Where a numeric range is recited, each intervening number within the range is explicitly contemplated with the same degree of precision. For example, for the range 6 to 9, the numbers 7 and 8 are contemplated in addition to 6 and 9, and for the range 6.0 to 7.0, the numbers 6.0, 6.1, 6.2, 6.3, 6.4, 6.5, 6.6, 6.7, 6.8, 6.9 and 7.0 are explicitly contemplated. In the same manner, all recited ratios also include all sub-ratios falling within the broader ratio.

The singular forms “a,” “an” and “the” include plural referents unless the context clearly dictates otherwise.

The term “about” generally refers to a range of numbers that one of skill in the art would consider equivalent to the recited value (i.e., having the same function or result). In many instances, the term “about” may include numbers that are rounded to the nearest significant figure.

As used herein, the term “Patient” refers to an adult human. The terms “patient” and “subject” are used interchangeably herein.

“Pharmaceutically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, mono-malic acid, mono oxalic acid, tartaric acid such as mono tartaric acid (e.g., (+) or (−)-tartaric acid or mixtures thereof), amino acids (e.g., (+) or (−)-amino acids or mixtures thereof), and the like. These salts can be prepared by methods known to those skilled in the art.

### Abbreviations

Ab: Antibody
ACR: American College of Rheumatology
ADL: Activities of daily living
AE: Adverse event
AESI: Adverse events of special interest
Ag: Antigen
ALT: Alanine transaminase
ANA: Antinuclear antibody
ANC: Absolute neutrophil count
anti-dsDNA: Anti-double stranded DNA
AO: As observed
AST: Aspartate transaminase
AUC: Area under the plasma/serum concentration-time curve
AUC/D: Dose-normalized AUC
AUC₀₋₂₄: Area under the plasma/serum concentration-time curve from time 0 to 24 hours
AUC₂₄: Area under the plasma/serum concentration-time curve at 24 hours
AUC_{inf}: Area under the plasma/serum concentration-time curve from time 0 to extrapolated to infinite time
AUCₛₛ: Area under the plasma/serum concentration-time curve during the dosing interval at steady state
AUCₜ: Area under the plasma/serum concentration-time curve from time 0 to time of last measurable concentration
axSpA: Axial spondyloarthritis
BCG: Bacilli Calmette-Guerin
BCR: B cell receptor
BCRP: Breast cancer resistance protein
bDMARD: Biologic disease-modifying antirheumatic drug
BL: Baseline
BMBT: Buccal mucosal bleed times
B/P: Blood to plasma ratio
BP: Blood pressure
CAC: Cardiovascular Adjudication Committee
Cardiac: Cardiovascular
CD: Crohn's disease
CIA: Collagen-induced arthritis
CIS: Carcinoma in-situ
CL/F: Apparent clearance
CLL: Chronic lymphocytic leukemia
CLASI: Cutaneous Lupus Erythematosus Disease Area and Severity Index
CLp: Total plasma clearance
CLr: Renal clearance
Cmax: Maximum observed serum or plasma concentration
Cmax/D: dose normalized Cmax
CMH: Cochran-Mantel-Haenszel
CNS: Central nervous system
COVID-19: Coronavirus disease 2019
CPK: Creatine Phosphokinase
CRF: Case report form
csDMARD: Conventional synthetic disease-modifying antirheumatic drug
CT: Computed tomography
CTCAE: Common Terminology Criteria for Adverse Events
CVA: Cerebrovascular accident
CXR: Chest x-ray
CYP3A: Cytochrome P450 3A isoform subfamily
CYP1A2: Cytochrome P450 1A2 isoform subfamily
CYP2B6: Cytochrome P450 2B6 isoform subfamily
CYP2C19: Cytochrome P450 2C19 isoform subfamily
DAP12L DNAX activating protein
DMC: Data Monitoring Committee
DNA: Deoxyribonucleic acid
dsDNA: Double stranded deoxyribonucleic acid
EC₅₀: Half-maximal effective concentration
ECG: Electrocardiogram
eCRF: Electronic case report form
Emax: Maximum induction effect determined in vitro
EU: European Union
EudraCT: European Clinical Trials Database
FACIT: Functional Assessment of Chronic Illness Therapy
FAS: Full analysis set
FcR: Fc receptor
FMO: Flavin containing monooxygenase
FSH: Follicle-stimulating hormone
Fu: Unbound fraction
GCA: Giant cell arteritis
GCP: Good clinical practice
GCSF: Granulocyte-colony stimulating factor
GFR: Glomerular filtration rate
GI: Gastrointestinal
GM-CSF: Granulocyte-macrophage colony-stimulating factor
GPVI: Glycoprotein VI
GSH: Glutathione
GVHD: Graft-Versus-Host Disease
HB: Hepatitis B
HBc Ab: Hepatitis B core antibody
HBs Ag: Hepatitis B surface antigen
HBV: Hepatitis B virus
HCV: Hepatitis C virus
HCV Ab: Hepatitis C virus antibody
hERG: Human ether-a-go-go-related gene
HIV: Human immunodeficiency virus
HIV Ab: HIV antibody
IC50: Inhibitory concentration producing 50% inhibition
ICH: International Council for Harmonisation
IEC: Independent ethics committee
IEC/IRB: Independent Ethics Committee/Institutional Review Board
IERC: Internal Executive Review Committee
IFN: Interferon
IgE: Immunoglobulin E
IgG: Immunoglobulin G
IgM: Immunoglobulin M
IM: Intramuscular
IMP: Investigational Medicinal Product
IRB: Institutional review board
IRT: Interactive response technology
IU: International Unit
IUD: Intrauterine device
IUS: Intrauterine hormone-releasing system
ITAM: Immunoreceptor tyrosine-based activation motif
IV: Intravenous
IVIG: Intravenous immunoglobulin
JAK: Janus kinase
JAK1: Janus kinase 1
JAK2: Janus kinase 2
JAK3: Janus kinase 3
JIA: Juvenile idiopathic arthritis
Km: Concentration at which the rate is ½ maximum
LLDAS: Lupus Low Disease Activity State
LTE: Long-term extension
LupusQoL: Lupus Quality of Life questionnaire
MACE: Major adverse cardiovascular event
MDCK-MDR1: Madin-Darby canine kidney cells transfected with the human MDR1 gene
MDRD: Modification of Diet in Renal Disease
MedDRA: Medical Dictionary for Regulatory Activities
MI: Myocardial infarction
MMRM: Mixed-effect Model Repeated Measures
MPV: Mean platelet volume
MTX: Methotrexate
N/A: Not applicable
NCI: National Cancer Institute
NMSC: Non-melanoma skin cancer
NRI-C: Non-Responder Imputation while incorporating Multiple Imputation to handle missing
data due to COVID-19
NRS: Numerical rating scale
NSAID: Nonsteroidal anti-inflammatory drug
OC: Observed Case
Papp: Apparent permeability coefficient
PBMC: Peripheral blood mononuclear cell
PCR: Polymerase chain reaction
PD: Premature Discontinuation
P-gp: P-glycoprotein
PhGA: Physician's Global Assessment
PI: Principal Investigator
PK: Pharmacokinetic(s)
PO: Per os (oral)
PPD: Purified protein derivative (tuberculin)
PRN: As needed
PRO: Patient-reported outcome
PtGA: Patient global assessment
QD: Once a day
QTc: QT interval corrected for heart rate
QTcF: QT interval corrected for heart rate using Fridericia's formula
QTcV: QT interval corrected with Van de Water's formula
RA: Rheumatoid arthritis
Rac: Accumulation ratio
RANK: Receptor activator of nuclear factor κ B
RBC: Red blood cell
RCT: Randomized clinical trial
REML: Restricted maximum likelihood
RNA: Ribonucleic acid
ROA: Route of administration
SAE: Serious adverse event
SAP: Statistical analysis plan
SAR: Serious adverse reaction
SELENA: Safety of Estrogens in Lupus Erythematosus National Assessment
SF-36: 36-Item Short Form Health Survey
SFI: SLEDAI Flare Index
SLE: Systemic lupus erythematosus
SLEDAI: Systemic Lupus Erythematosus Disease Activity Index
SLEDAI-2K: Systemic Lupus Erythematosus Disease Activity Index 2000
SJC: Swollen joint count
SLICC: Systemic Lupus Erythematosus International Collaborating Clinics
Sm/RNP: Smith/ribonucleoprotein
SRI: SLE Responder Index
SSc: Systemic sclerosis
SUSAR: Suspected unexpected serious adverse reaction
Syk: Spleen tyrosine kinase
TA MD: Therapeutic Area MD
TB: Tuberculosis
TBNK: T lymphocytes, B lymphocytes, and natural killer lymphocytes
TEAE: Treatment-emergent adverse event
TJC: Tender joint count
TNF: Tumor necrosis factor
trFRET: Time resolved fluorescence resonance energy transfer
UC: Ulcerative colitis
UK: United Kingdom
ULN: Upper limit of normal
UPCR: Urine protein to creatinine ratio
US: United States
vs: versus
Vss/F: Apparent volume of distribution at steady state
WBC: White blood cells
WOCBP: Women of Childbearing Potential

### Endpoint Definitions

SLE Responder Index (SRI)-4 is defined as follows with all criteria compared to Baseline:
≥4-point reduction in Systemic Lupus Erythematosus Disease Activity Index 2000 (SLEDAI-2K) score.
No worsening in PhGA, i.e., <0.3 points increase relative to Baseline on a 3-point Physician's Global Assessment (PGA) visual analogue scale (VAS).
No new British Isles Lupus Assessment Group (BILAG) A or more than 1 new BILAG B disease activity scores (i.e., no organ system changes from Baseline B/C/D/E to A and no more than 1 organ system changes from Baseline C/D/E to B).

BILAG-Based Combined Lupus Assessment (BICLA) is defined as follows:
Improvement in organ systems:
   Improvement in all initial (Baseline) A and B scores, i.e., all BILAG A scores at Baseline improved to B or C or D, and all BILAG B scores at Baseline improved to C or D.
   No new BILAG A or more than one new BILAG B score compared to Baseline (i.e., no organ system changes from Baseline B/C/D/E to A and no more than 1 organ system changes from Baseline C/D/E to B).
No worsening of the overall condition, i.e., <0.3 point increase in PhGA relative to Baseline.
Improvement in disease activity: no increase of SLEDAI-2K total score from Baseline.

Lupus Low Disease Activity State (LLDAS) is defined as follows:
SLEDAI-2K score ≤4.
No activity in major organ systems: defined as no category A or B in any BILAG system except Mucocutaneous and Musculoskeletal.
No new lupus disease activity compared with the previous assessment: new lupus disease activity compared with the previous assessment is defined as category A or B in any BILAG system where an item score of ‘New’ or ‘Worse’ that leads to an A or B category.
PhGA ≤1 (0-3 scale).
Steroid dose ≤7.5 mg daily.
Well-tolerated standard maintenance doses of immunosuppressive drugs and approved biological agents, excluding investigational drugs. Defined as no increase in concomitant immunosuppressant and antimalarial drugs above baseline dose

### II. JAK1 Inhibition

Upadacitinib (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide), or a pharmaceutically acceptable salt or solid state form thereof, is an oral Janus kinase (JAK) inhibitor that displays unique selectivity for the JAK1 receptor. Upadacitinib has the structure shown below:

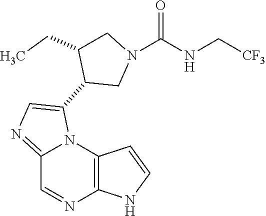 [SMILES: CC[C@@H]1CN(C(=O)NCC(F)(F)F)C[C@@H]1c1cnc2cnc3[nH]ccc3n12]

The dosage strength of upadacitinib recited in the present application is based on the weight of anhydrous freebase upadacitinib present in the active ingredient delivered to the patient. For example, a dose of “15 mg of upadacitinib” or “UPA 15 MG” refers to the 15 mg amount of the neutral upadacitinib freebase present in the active ingredient, not including any coformer (e.g., solvent or water molecule(s)) of a solvate or hydrate (including hemihydrate) or counteranions of a pharmaceutically acceptable salt), that may also be present in the active ingredient. So, for example, the administration of “15 mg of upadacitinib” includes the administration of 15.4 mg of crystalline upadacitinib freebase hemihydrate (which includes ½ of a water conformer molecule per upadacitinib freebase molecule) which delivers 15 mg of anhydrous freebase upadacitinib to a patient.

The dosage strength of upadacitinib recited in the present application is based on the weight of anhydrous freebase upadacitinib present in the active ingredient delivered to the patient. For example, a dose of “30 mg of upadacitinib” or “UPA 30 MG” refers to the 30 mg amount of the neutral upadacitinib freebase present in the active ingredient, not including any coformer (e.g., solvent or water molecule(s)) of a solvate or hydrate (including hemihydrate) or counteranions of a pharmaceutically acceptable salt), that may also be present in the active ingredient. So, for example, the administration of “30 mg of upadacitinib” includes the administration of 30.7 mg of crystalline upadacitinib freebase hemihydrate (which includes ½ of a water conformer molecule per upadacitinib freebase molecule) which delivers 30 mg of anhydrous freebase upadacitinib to a patient.

The dosage strength of upadacitinib recited in the present application is based on the weight of anhydrous freebase upadacitinib present in the active ingredient delivered to the patient. For example, a dose of “45 mg of upadacitinib” or “UPA 45 MG” refers to the 45 mg amount of the neutral upadacitinib freebase present in the active ingredient, not including any coformer (e.g., solvent or water molecule(s)) of a solvate or hydrate (including hemihydrate) or counteranions of a pharmaceutically acceptable salt), that may also be present in the active ingredient. So, for example, the administration of “45 mg of upadacitinib” includes the administration of 46.1 mg of crystalline upadacitinib freebase hemihydrate (which includes ½ of a water conformer molecule per upadacitinib freebase molecule) which delivers 45 mg of anhydrous freebase upadacitinib to a patient.

### III. Treatment of Systemic Lupus Erythematosus with Upadacitinib

As described herein above, upadacitinib has anti-inflammatory properties, and accordingly, is predicted according to the present disclosure to be effective in decreasing signs and symptoms associated with active SLE in patients. Particularly, Janus kinase 1 (JAK1) inhibition via upadacitinib is expected to disrupt T cell activation and Type I interferon (IFN) signaling. Without wishing to be bound by theory, it is believed that inhibition of JAK1 will result in amelioration of lupus disease activity, and treatment with upadacitinib has the potential to provide greater clinical efficacy compared to current SLE standard of care while maintaining an acceptable safety profile. To explore the safety, tolerability, and efficacy of the upadacitinib in SLE, several Phase I and II clinical studies have been conducted.

Accordingly, in one aspect is provided a method of treating a human patient having systemic lupus erythematosus. In some embodiments, provided is a method of treating a human patient having systemic lupus erythematosus who is receiving standard therapy. In some embodiments, provided is a method of treating a human patient having moderately to severely active systemic lupus erythematosus. In some embodiments, provided is a method of treating a human patient having moderately to severely active systemic lupus erythematosus who is receiving standard therapy. In some embodiments, provided is a method of treating a human patient with moderate to severe systemic lupus erythematosus. In some embodiments, provided is a method of treating a human patient with moderate to severe systemic lupus erythematosus who is receiving standard therapy. In some embodiments, provided is a method of treating a human patient with active, auto-antibody positive systemic lupus erythematosus. In some embodiments, provided is a method of treating a human patient with active, auto-antibody positive systemic lupus erythematosus who is receiving standard therapy.

In some embodiments, the standard therapy the patient is receiving includes a corticosteroid, an antimalarial agent, or a combination thereof. In some embodiments, the standard therapy includes prednisone (or prednisone equivalent) and an antimalarial agent. In some embodiments, the standard therapy includes an antimalarial agent, such as hydroxychloroquine.

In some embodiments, the standard therapy further includes azathioprine, mycophenolate, leflunomide, cyclosporine, tacrolimus, methotrexate or a combination thereof.

In another aspect is provided a method of treating a human patient having moderately to severely active systemic lupus erythematosus, the method comprising orally administering once daily to the patient 30 mg of upadacitinib.

In another aspect is provided a method of treating a human patient having moderately to severely active systemic lupus erythematosus, the method comprising orally administering once daily to the patient 15 mg of upadacitinib.

In another aspect is provided a method of treating a human patient having moderately to severely active systemic lupus erythematosus, the method comprising orally administering once daily to the patient 45 mg of upadacitinib.

In yet another aspect is provided a method of treating a human patient having moderately to severely active systemic lupus erythematosus, the method comprising orally administering once daily to the patient an induction dose of upadacitinib for a period of time. In some embodiments, the induction dose is 45 mg, administered once daily.

In some embodiments, the induction dose is followed by administration of a maintenance dose. In some embodiments, the maintenance dose is 30 mg. In some embodiments, the maintenance dose is 15 mg.

Accordingly, in some embodiments, the method comprises orally administering once daily to the patient a 45 mg induction dose of upadacitinib for a period of time, followed by orally administering once daily to the patient a maintenance dose of 30 mg or 15 mg.

In some embodiments, the method results in a SLE Responder Index (SRI)-4 response and Prednisone Equivalent Steroid Dose ≤10 mg at 52 weeks after the first daily administration.

In some embodiments, the method results in a SLE Responder Index (SRI)-4 response at 52 weeks after the first daily administration.

In some embodiments, the method results in a BILAG-Based Combined Lupus Assessment (BICLA) response at 52 weeks after the first daily administration.

In some embodiments, the method results in a Lupus Low Disease Activity State (LLDAS) response at 52 weeks after the first daily administration.

In some embodiments, the method results in a reduction in steroid burden, assessed as change from Baseline in prednisone equivalent steroid dose at Week 52.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 52.

In some embodiments, the method results in a SLE Responder Index (SRI)-4 response and Prednisone Equivalent Steroid Dose 10 mg at 48 weeks after the first daily administration.

In some embodiments, the method results in a SLE Responder Index (SRI)-4 response at 48 weeks after the first daily administration.

In some embodiments, the method results in a BILAG-Based Combined Lupus Assessment (BICLA) response at 48 weeks after the first daily administration.

In some embodiments, the method results in a Lupus Low Disease Activity State (LLDAS) response at 48 weeks after the first daily administration.

In some embodiments, the method results in a reduction in steroid burden, assessed as change from Baseline in prednisone equivalent steroid dose at Week 48.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 48.

In some embodiments, the method results in a SLE Responder Index (SRI)-4 response and Prednisone Equivalent Steroid Dose ≤10 mg at 24 weeks after the first daily administration.

In some embodiments, the method results in a SLE Responder Index (SRI)-4 response at 24 weeks after the first daily administration.

In some embodiments, the method results in a BILAG-Based Combined Lupus Assessment (BICLA) response at 24 weeks after the first daily administration.

In some embodiments, the method results in a Lupus Low Disease Activity State (LLDAS) response at 24 weeks after the first daily administration.

In some embodiments, the method results in a reduction in steroid burden, assessed as change from Baseline in prednisone equivalent steroid dose at Week 24.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 24.

In some embodiments, the method results in a SLE Responder Index (SRI)-4 response and Prednisone Equivalent Steroid Dose 10 mg at 20 weeks after the first daily administration.

In some embodiments, the method results in a SLE Responder Index (SRI)-4 response at 20 weeks after the first daily administration.

In some embodiments, the method results in a BILAG-Based Combined Lupus Assessment (BICLA) response at 20 weeks after the first daily administration.

In some embodiments, the method results in a Lupus Low Disease Activity State (LLDAS) response at 20 weeks after the first daily administration.

In some embodiments, the method results in a reduction in steroid burden, assessed as change from Baseline in prednisone equivalent steroid dose at Week 20.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 20.

In some embodiments, the method results in a SLE Responder Index (SRI)-4 response and Prednisone Equivalent Steroid Dose 10 mg at 16 weeks after the first daily administration.

In some embodiments, the method results in a SLE Responder Index (SRI)-4 response at 16 weeks after the first daily administration.

In some embodiments, the method results in a BILAG-Based Combined Lupus Assessment (BICLA) response at 16 weeks after the first daily administration.

In some embodiments, the method results in a Lupus Low Disease Activity State (LLDAS) response at 16 weeks after the first daily administration.

In some embodiments, the method results in a reduction in steroid burden, assessed as change from Baseline in prednisone equivalent steroid dose at Week 16.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 16.

In some embodiments, the method results in a SLE Responder Index (SRI)-4 response and Prednisone Equivalent Steroid Dose ≤10 mg at 12 weeks after the first daily administration.

In some embodiments, the method results in a SLE Responder Index (SRI)-4 response at 12 weeks after the first daily administration.

In some embodiments, the method results in a BILAG-Based Combined Lupus Assessment (BICLA) response at 12 weeks after the first daily administration.

In some embodiments, the method results in a Lupus Low Disease Activity State (LLDAS) response at 12 weeks after the first daily administration.

In some embodiments, the method results in a reduction in steroid burden, assessed as change from Baseline in prednisone equivalent steroid dose at Week 12.

In some embodiments, the p method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 12.

In some embodiments, the method results in a SLE Responder Index (SRI)-4 response and Prednisone Equivalent Steroid Dose 10 mg at 8 weeks after the first daily administration.

In some embodiments, the method results in a SLE Responder Index (SRI)-4 response at 8 weeks after the first daily administration.

In some embodiments, the method results in a BILAG-Based Combined Lupus Assessment (BICLA) response at 8 weeks after the first daily administration.

In some embodiments, the method results in a Lupus Low Disease Activity State (LLDAS) response at 8 weeks after the first daily administration.

In some embodiments, the method results in a reduction in steroid burden, assessed as change from Baseline in prednisone equivalent steroid dose at Week 8.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 8.

In some embodiments, the method results in a SLE Responder Index (SRI)-4 response and Prednisone Equivalent Steroid Dose ≤10 mg at 4 weeks after the first daily administration.

In some embodiments, the method results in a SLE Responder Index (SRI)-4 response at 4 weeks after the first daily administration.

In some embodiments, the method results in a BILAG-Based Combined Lupus Assessment (BICLA) response at 4 weeks after the first daily administration.

In some embodiments, the method results in a Lupus Low Disease Activity State (LLDAS) response at 4 weeks after the first daily administration.

In some embodiments, the method results in a reduction in steroid burden, assessed as change from Baseline in prednisone equivalent steroid dose at Week 4.

In some embodiments, the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 4.

In some embodiments, the method results in a significant decrease in the levels of anti-dsDNA autoantibodies and complement C3 and C4. In some embodiments, the method results in a significant increase in the numbers of circulating memory, naïve, and total B cells.

In some embodiments, the patient has had an inadequate response or intolerance to one or more disease-modifying antirheumatic drugs. In some embodiments, the patient has had an inadequate response or intolerance to methotrexate. In some embodiments, the patient has had an inadequate response or intolerance to an anti-TNF biologic agent. In some embodiments, the patient has had an inadequate response or intolerance to an anti-malarial agent.

In some embodiments, the patient has had an inadequate response or intolerance to an immunomodulator.

In some embodiments, the patient has had an inadequate response or intolerance to a corticosteroid.

In some embodiments, the patient has had an inadequate response or intolerance to prednisone (or prednisone equivalent), an antimalarial agent, azathioprine, mycophenolate, leflunomide, cyclosporine, tacrolimus, or methotrexate.

In some embodiments, the patient is concomitantly treated with an anti-malarial agent.

In some embodiments, the patient is concomitantly treated with an immunomodulator.

In some embodiments, the patient is concomitantly treated with a corticosteroid.

In some embodiments, the patient is concomitantly treated with Methotrexate. In some embodiments, the patient is concomitantly treated with 20 mg/wk Methotrexate with concomitant folic acid ≤5 mg/wk.

In some embodiments, the patient is concomitantly treated with Azathioprine. In some embodiments, the patient is concomitantly treated with ≤150 mg/day Azathioprine.

In some embodiments, the patient is concomitantly treated with Mycophenolate mofetil.

In some embodiments, the patient is concomitantly treated with ≤2,000 mg/day Mycophenolate mofetil.

In some embodiments, the patient is concomitantly treated with Mycophenolate sodium.

In some embodiments, the patient is concomitantly treated with ≤1,440 mg/day Mycophenolate sodium.

In some embodiments, the patient is concomitantly treated with Hydroxychloroquine. In some embodiments, the patient is concomitantly treated with ≤400 mg/day Hydroxychloroquine.

In some embodiments, the patient is concomitantly treated with Chloroquine. In some embodiments, the patient is concomitantly treated with ≤500 mg/day Chloroquine.

In some embodiments, the patient is concomitantly treated with Quinacrine. In some embodiments, the patient is concomitantly treated with ≤100 mg/day Quinacrine.

In some embodiments, the patient is concomitantly treated with Leflunomide. In some embodiments, the patient is concomitantly treated with ≤20 mg/day Leflunomide.

In some embodiments, the patient is concomitantly treated with Cyclosporine. In some embodiments, the patient is concomitantly treated with Cyclosporine dosed by serum levels.

In some embodiments, the patient is concomitantly treated with Tacrolimus. In some embodiments, the patient is concomitantly treated with Tacrolimus dosed by serum levels.

In some embodiments, the patient is concomitantly treated with corticosteroids (prednisone-equivalent). In some embodiments, the patient is concomitantly treated with ≤20 mg/day corticosteroids, decreased no faster than 5 mg QD per week. In some embodiments, corticosteroids are increased by no more than 5 mg QD per week regardless of Baseline dose, to a maximum dose of 25 mg, through Week 8 of treatment.

### IV. Pharmaceutical Compositions and Routes of Administration

Upadacitinib can be administered to a human patient by itself or in pharmaceutical composition where it is mixed with biologically suitable carriers or excipient(s) at doses to treat or ameliorate a disease or condition as described herein. Mixtures of these compounds can also be administered to the patient as a simple mixture or in suitable formulated pharmaceutical compositions.

The pharmaceutical compositions of the present disclosure may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

### EXEMPLIFICATION

### Example 1: Safety and Efficacy of Upadacitinib in Systemic Lupus Erythematosus (SLE)

This was a Phase 2 Study (M19-130) to Investigate the Safety and Efficacy of Upadacitinib Given in Subjects with Moderately to Severely Active Systemic Lupus Erythematosus.

### Study Population and Number of Subjects to be Enrolled

Approximately 130 adult male or female subjects who were 18 to 65 years of age, inclusive, and had a clinical diagnosis of SLE at least 24 weeks prior to Screening, meeting at least 4 of the 11 revised Criteria for Classification of SLE according to the 1997 Update of the 1982 American College of Rheumatology (ACR) OR meeting at least 4 of the 2012 Systemic Lupus Erythematosus International Collaborating Clinics (SLICC) classification criteria, including at least 1 clinical criterion and 1 immunologic criterion. Subjects must have had active SLE by SLEDAI-2K ≥6 as reported and independently adjudicated (clinical score ≥4, excluding lupus headache and/or organic brain syndrome) at Screening and Baseline. If 4 points of the required entry points were for arthritis, there must also have been a minimum of 3 tender and 3 swollen joints.

### Efficacy Endpoints

### Primary Endpoint

The primary efficacy endpoint was achievement of SLE Responder Index (SRI)-4 and steroid dose ≤10 mg prednisone equivalent once a day (QD) at Week 24. SLE Responder Index (SRI)-4 is defined as ≥4-point reduction in Systemic Lupus Erythematosus Disease Activity Index 2000 (SLEDAI-2K) score without worsening of the overall condition (no worsening in Physician's Global Assessment [PhGA], <0.3 point increase) or the development of significant disease activity in new organ systems (no new British Isles Lupus Assessment Group ([BILAG]) A or >1 new BILAG B).

### Secondary Endpoints

The following secondary efficacy endpoints were evaluated:
1. Achievement of SRI-4 at Week 24
2. Achievement of BILAG-Based Combined Lupus Assessment (BICLA) response at Week 24
3. Achievement of Lupus Low Disease Activity State (LLDAS) at Week 24
4. Steroid burden, assessed as change from Baseline at Week 24
5. Number of mild, moderate, or severe flares per patient-year (respectively and overall) by Safety of Estrogens in Lupus Erythematosus National Assessment (SELENA) SLEDAI Flare Index (SFI), assessed by number and types of flare per subject through Week 24

### Additional Endpoints

The following additional efficacy endpoints were evaluated at all visits unless otherwise noted:
1. Achievement of SRI-4
2. Achievement of SRI-5, -6, -7, -8 and steroid dose ≤10 mg prednisone equivalent QD at Weeks 24 and 48
3. Achievement of SRI-5, -6, -7, -8
4. Achievement of BICLA response
5. Achievement of LLDAS
6. Achievement of ≥4-point decrease in SLEDAI-2K from Baseline
7. Steroid burden, assessed as change from Baseline
8. Number of mild, moderate, or severe flares per patient-year (respectively and overall) by SELENA SFI, assessed by number and types of flare per subject through Week 24 and Week 48
9. Time to first flare by SELENA SFI after first study drug administration up to Week 24 and Week 48
10. Achievement of 50% reduction of tender or swollen lupus joints defined as ≥50% decrease in either tender or swollen joints (of those starting with total ≥6 affected joints)
11. Achievement of 50% reduction in Cutaneous Lupus Erythematosus Disease Area and Severity Index (CLASI) activity score (of those starting with CLASI ≥10)
12. Change in SLEDAI-2K from Baseline
13. Change in BILAG from Baseline
14. Change in PhGA from Baseline
15. Change in Patient Global Assessment (PtGA) from Baseline
16. Change from Baseline in Functional Assessment of Chronic Illness Therapy (FACIT)—Fatigue at Weeks 2, 12, 24, and 48
17. Change from Baseline in 36-Item Short Form Health Survey (SF-36) at Weeks 2, 12, 24, and 48
18. Change from Baseline Lupus Quality of Life questionnaire (LupusQoL) at Weeks 2, 12, 24, and 48
19. Change from Baseline Pain Numerical Rating Scale (NRS) at Weeks 2, 12, 24, and 48

### Safety Endpoints

Routine safety evaluations included adverse event (AE) monitoring, physical examinations, vital sign measurements, electrocardiograms (ECGs), and clinical laboratory testing (hematology, chemistry, and urinalysis as a measure of safety and tolerability for the entire study duration. SAEs were assessed at any dose that resulted in a death, inpatient hospitalization or prolongation of existing hospitalization, persistent or significant disability/incapacity, or a congenital anomaly.

The following areas of safety interest were routinely assessed to identify any major safety findings related to immunosuppression or potential risks associated with the individual classes of therapy: serious and/or opportunistic infections, herpes zoster, active TB, malignancies (all types), adjudicated gastrointestinal (GI) perforations, adjudicated cardiovascular events (e.g., major adverse cardiovascular event [MACE]), anemia, neutropenia, lymphopenia, renal dysfunction, hepatic disorders, and adjudicated embolic and thromboembolic events (non-cardiac, non-central nervous system [CNS]) including venous thrombotic events defined as pulmonary embolism and deep vein thrombosis. Specific toxicity management measures were utilized for serious infections, serious GI events, cardiovascular events (MACE), malignancies, ECG abnormalities, and select laboratory abnormalities. In addition, a 30-day follow-up phone call from the last dose of study drug occurred to determine the status of any ongoing AEs/SAEs, or the occurrence of any new AEs/SAEs. An unblinded internal DMC was established to ensure the overall integrity and conduct of the study. DMC reviews were conducted at regular intervals. Regular systematic reviews of emerging safety data from all clinical studies with upadacitinib was conducted by AbbVie. An independent external Cardiovascular Adjudication Committee (CAC) adjudicated all blinded cardiovascular and cerebrovascular events, embolic/thrombotic events, and deaths, as defined in the CAC charter.

### Pharmacokinetic Endpoints

Upadacitinib plasma concentrations were summarized at each sampling time point using descriptive statistics. A mixed-effects modeling approach may have been used to estimate the population central value and the empirical Bayesian estimates of the individual values for upadacitinib oral clearance (CL/F) and volume of distribution (Vss/F). Additional parameters and pharmacokinetic (PK)/pharmacodynamic relationships may have been estimated if useful in the interpretation of the data.

### Biomarker Research

Biospecimens (whole blood, peripheral blood mononuclear cells [PBMCs], plasma, and serum) were collected at specified time points throughout the study to evaluate known and/or novel disease-related or drug-related biomarkers. Types of biomarkers may have included nucleic acids, proteins, lipids, RNA, DNA, and/or metabolites. The objective of this research was to analyze samples for biomarkers that would help to understand SLE, related conditions, and response to treatment with upadacitinib or similar compounds.

Type I IFN is a key driver of disease pathogenesis in lupus, and high IFN signature is associated with active SLE. Approximately 60% of lupus patients express an elevated Type I IFN gene signature in the blood, suggesting higher Type I IFN activity in these individuals. JAK1 inhibition is expected to disrupt Type I IFN signaling; therefore, lupus patients expressing the Type I gene signature may be more likely to benefit from treatment with upadacitinib. A Type I IFN RNA signature consisting of 4 genes (IFI6, IFI27, IFIT1, and MX1) was evaluated using a validated assay with a cut point set at ≥−0.25 for IFN high vs <−0.25 for IFN low (−0.25 is the upper limit of the cut point) using the Type I IFN Gene Expression Assay Score.

In the event an IFN score could not be collected or analyzed at Screening due to COVID-19 lab access, that subject was still be enrolled in the study and assigned a score of Not Applicable for the Screening visit. Samples drawn at the Week 4 and Week 24 visits may have been used for analysis, if collected.

These results robustly dichotomize the IFN high and low population. The assay was performed on Screening samples and at subsequent time points unless precluded by local regulations or restrictions (IFN signature samples were not collected at sites in China). Screening values were used as a criterion for subject enrichment with the goal of enrolling 80% of the study subjects with high composite core. Stratifying the study to include approximately 80% high IFN score population allowed for increased confidence in the subject population.

Research may also have included analysis of genes, epigenetics, gene expression, and proteomic biomarkers that may associate with SLE, related conditions, or the subject's response to treatment. This research was exploratory in nature and the results may not have been included with the clinical study report. Required biomarker samples were collected and analyzed from all subjects, unless precluded by local regulations or restrictions (for such cases, optionality of collection/analysis is given through an appropriate Informed Consent Form).

### Investigational Plan

The study was designed to investigate the safety and efficacy of upadacitinib in subjects with moderately to severely active SLE despite standard of care therapy. The study duration included a 42-day maximum screening period, a 48-week randomized, placebo-controlled, double-blind, parallel-group treatment period, and a 30-day follow-up phone call. The Week 24 Primary Analysis was performed when all subjects completed the Week 24 visit or discontinued the study. Sites and subjects remained blinded throughout the study. The study team remained blinded until the Week 24 Primary Analysis. Study visits were conducted at Screening, Baseline, and Weeks 2, 4, 8, 12, 16, 20, 24, 28, 32, 36, 40, 44, and 48/Premature Discontinuation (PD). A 30-day follow-up phone call from the last dose of study drug occurred to determine the status of any ongoing adverse events (AEs)/serious adverse events (SAEs) or the occurrence of any new AEs/SAEs.

A long-term extension (LTE) study is being conducted under a separate protocol (LTE Study M20-186) at sites where it was permitted by the local Competent Authority and Ethics Committee. Subject rollover from the initial randomized portion of the study occurred at Week 48. A planned unblinded interim analysis was performed when 50% of the planned subjects completed their Week 24 assessments. The objective of this analysis was to reassess the treatment regimens in Study M19-130 and the benefit/risk for rollover into LTE Study M20-186. The interim analysis was performed by an independent team at AbbVie that is separate and apart from the blinded study team. The Study M19-130 team remained blinded through the Week 24 Primary Analysis.

### Key Eligibility Criteria

The following were the key eligibility criteria for the study:
Adult male or female, 18 to 65 years of age, inclusive, at Screening.
Clinical diagnosis of SLE at least 24 weeks prior to Screening, meeting at least 4 of the 11 revised Criteria for Classification of SLE according to the 1997 Update of the 1982 ACR OR meeting at least 4 of the 2012 SLICC classification criteria, including at least 1 clinical criterion and 1 immunologic criterion.
At Screening, must have had at least one of the following:
   Anti-nuclear antibody test (ANA)+(titer ≥1:80)
   Anti-double stranded DNA (anti-dsDNA)+
   anti-Smith+
   SLEDAI-2K ≥6 despite background therapy as reported and independently adjudicated (clinical score ≥4, excluding lupus headache and/or organic brain syndrome) at Screening. If 4 points of the required entry points are for arthritis, there must also have been a minimum of 3 tender and 3 swollen joints. If subject had rash and Principal Investigator (PI) considered it to be attributable to SLE, subject consented to skin photograph collection for adjudication (re-confirmed at Baseline visit).
   PhGA ≥1 during screening period.
Must have been on background treatment, stable for 30 days, at Baseline, and throughout the study, with prednisone (or prednisone equivalent) (≤20 mg), antimalarials, azathioprine (≤150 mg), mycophenolate (2 g), leflunomide (20 mg), cyclosporine, tacrolimus, and/or methotrexate (MTX) (20 mg).
No combinations of the above with immunomodulators other than prednisone (or equivalents) and antimalarials was permitted. Must not have been using intravenous (IV) or intramuscular (IM) corticosteroids greater than or equal to a 40 mg prednisone-equivalent.

### Study Drug and Duration of Treatment

Study drug was taken orally as 1 film-coated tablet containing 30 mg of upadacitinib and/or matching placebo once daily with or without food for 24 weeks.

Overall Study Design and Plan

This was a multi-center, randomized, double-blind, and placebo-controlled Phase 2 study, with a Week 24 primary endpoint, to investigate the safety and efficacy of upadacitinib in subjects with moderately to severely active SLE. The study was continued through Week 48 to further assess maintenance of efficacy and safety of each treatment beyond 24 weeks.

Approximately 130 male and female subjects, 18 to 65 years of age, inclusive, with a diagnosis of moderately to severely active SLE (clinical diagnosis of SLE at least 24 weeks prior to Screening, meeting at least 4 of the 11 revised Criteria for Classification of SLE according to the 1997 Update of the 1982 American College of Rheumatology [ACR] OR meeting at least 4 of the 2012 Systemic Lupus Erythematosus International Collaborating Clinics [SLICC] classification criteria, including at least 1 clinical criterion and 1 immunologic criterion) despite standard of care therapy were be randomized as follows:
Group 1: upadacitinib 30 mg QD (n=65)
Group 2: upadacitinib placebo QD (n=65)

Randomization was stratified based on the following factors:
Baseline corticosteroid dose above 10 mg prednisone-equivalent (≤10 mg or >10 mg)
Screening SLEDAI-2K (<10 or ≥10)
High vs low vs not applicable IFN score (approximately 80% high) (this stratification factor did not apply to subjects in China)
Baseline immunosuppressant (azathioprine, tacrolimus, cyclosporine, methotrexate [MTX], mycophenolate, or leflunomide) (yes/no)

The study duration included a 42-day maximum screening period and a 48-week randomized, placebo-controlled, double-blind, parallel-group treatment period with a 30-day follow-up phone call. Sites and subjects remained blinded throughout the study. The Week 24 Primary Analysis was performed when all subjects completed the Week 24 visit or had discontinued the study. The study team remained blinded until the Week 24 Primary Analysis. A 30-day follow-up phone call from the last dose of study drug occurred to determine the status of any ongoing AEs/SAEs or the occurrence of any new AEs/SAEs.

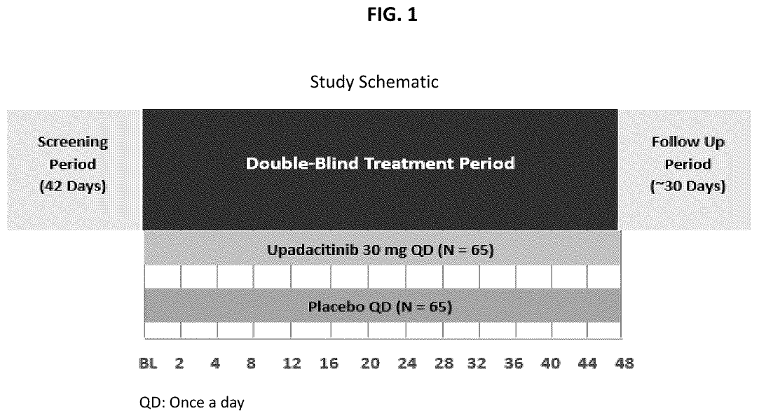

A schematic illustration of the study is shown in FIG. 1. A long-term extension (LTE) study was conducted under a separate protocol (LTE Study M20-186) at sites where it was permitted by the local Competent Authority and Ethics Committee. Subject rollover from the initial randomized portion of the study occurred at Week 48. When 50% of the planned subjects completed the Week 24 assessments, an unblinded interim analysis was performed by an independent team at AbbVie.

### Choice of Control Group

This was a parallel-group study consisting of treatment groups receiving upadacitinib or matching placebo to assess the safety and efficacy of upadacitinib once daily for 24 weeks in subjects with moderately or severely active SLE. Placebo served as a reference for efficacy assessment.

### Appropriateness of Measurements

All efficacy and safety measurements in this study were standard for assessing disease activity in subjects with SLE being treated with immunosuppressant therapies. The first safety evaluation was at Week 2, and at all subsequent visits. All clinical and laboratory procedures in this study were standard and generally accepted.

### Suitability of Subject Population

Eligible for this study were adult male or female subjects, 18 to 65 years of age, inclusive, with a clinical diagnosis of SLE at least 24 weeks prior to Screening, meeting at least 4 of the 11 revised Criteria for Classification of SLE according to the 1997 Update of the 1982 ACR OR meeting at least 4 of the 2012 SLICC classification criteria, including at least 1 clinical criterion and 1 immunologic criterion, and at least one of the following at Screening: positive antinuclear antibody (ANA) (titer ≥1:80), anti-double stranded DNA (dsDNA), or anti-Smith antibodies. The selection criteria identified individuals with active SLE that were the intended target population for treatment with upadacitinib, based on the current knowledge of the drug.

### Eligibility Criteria

Subjects must have met all of the following criteria in order to be included in the study. Anything other than a positive response to the questions below resulted in exclusion from study participation.

### Demographic and Laboratory Assessments

1. Adult male or female, 18 to 65 years of age, inclusive, at Screening.
2. Must not have laboratory values meeting the following criteria within the screening period prior to the first dose of study drug:
   Serum aspartate transaminase (AST) >2.0× upper limit of normal (ULN);
   Serum alanine transaminase (ALT) >2.0×ULN;
   Estimated glomerular filtration rate (GFR) by simplified 4-variable Modification of Diet in Renal Disease (MDRD) formula <40 mL/min/1.73 m²;
   Absolute neutrophil count (ANC)<1,000/μL;
   Platelet count <50,000/μL;
   Hemoglobin <9 g/dL.
3. Must not have positive titers for all 3 antiphospholipid antibodies known to be associated with venous thrombotic events at Screening: lupus anticoagulant, anti-beta 2 glycoprotein 1, and anticardiolipin antibody.

### Disease Activity

1. Clinical diagnosis of SLE at least 24 weeks prior to Screening, meeting at least 4 of the 11 revised Criteria for Classification of SLE according to the 1997 Update of the 1982 ACR OR meeting at least 4 of the 2012 SLICC classification criteria, including at least 1 clinical criterion and 1 immunologic criterion.
2. At Screening, must have at least one of the following:
   ANA+ (titer ≥1:80)
   anti-dsDNA+
   anti-Smith+
3. SLEDAI-2K ≥6 despite background therapy as reported and independently adjudicated (clinical score ≥4, excluding lupus headache and/or organic brain syndrome) at Screening:
   If 4 points of the required entry points are for arthritis, there must also be a minimum of three tender and three swollen joints.
   If 4 points of the required clinical score are for proteinuria attributable to active lupus nephritis, it must be >0.5 g/day equivalent (0.5 mg/mg).
   If subject has rash and Principal Investigator (PI) considers it to be attributable to SLE, subject must consent to skin photograph collection for adjudication.
   Score must be re-confirmed at the Baseline visit.
4. PhGA ≥1 during screening period.
5. The subject must be on background treatment throughout the study. The background treatment must be stable for 30 days prior to Baseline and throughout the study with
   Antimalarial(s), prednisone (or prednisone-equivalent) (≤20 mg), azathioprine (≤150 mg), mycophenolate (≤2 g), leflunomide (≤20 mg), cyclosporine, tacrolimus, and/or MTX (≤20 mg).
   The combination of background treatment with antimalarial(s) and/or prednisone (or equivalent) is permitted, and a single, but not multiple, additional immunosuppressant from the list above, is permitted.

### Subject History

1. Must not have active lupus nephritis (progressive Class IV or >1 g/day equivalent [1 mg/mg] proteinuria) or have undergone induction therapy within the last 6 months.
2. Must not be receiving hemodialysis (or other forms of renal replacement therapy).
3. Must not have active neuropsychiatric SLE as defined by the CNS portion of SLEDAI-2K or BILAG (excluding lupus headache).
4. Must not have any active, chronic, or recurrent viral or bacterial infection that, based on the Investigator's clinical assessment, makes the subject an unsuitable candidate for the study, including hepatitis B virus (HBV) or hepatitis C virus (HCV), recurrent or disseminated (even a single episode) herpes zoster, disseminated (even a single episode) herpes simplex, or HIV. Active HBV, HCV, and HIV are defined as:
   HBV: hepatitis B surface antigen (HBs Ag) positive (+) or detected sensitivity on the HBV DNA polymerase chain reaction (PCR) qualitative test for hepatitis B core antibody (HBc Ab) positive (+) subjects; (and for hepatitis B surface antibody [HBs Ab] positive [+] subjects in China and Japan only).
5. Must not have active TB or latent TB and did not complete a full course of prophylaxis for latent TB.
6. Must not have a history of any malignancy except for successfully treated non-melanoma skin cancer (NMSC) or localized carcinoma in-situ (CIS) of the cervix.
7. Must not have a history of clinically significant (per Investigator's judgment) drug or alcohol abuse within the last 6 months.
8. Must not have a history of GI perforation (other than appendicitis or penetrating injury), diverticulitis, or significantly increased risk for GI perforation per investigator judgment.
9. Must not have any conditions that could interfere with drug absorption including but not limited to short bowel syndrome (e.g., subjects with a history of gastric bypass surgery are excluded).

Subjects with a history of gastric banding/segmentation are not excluded.
10. Must not be a recipient of an organ transplant.
11. Must not have history of clinically significant medical conditions or any other reason that in the opinion of the Investigator would interfere with the subject's participation in this study or would make the subject an unsuitable candidate to receive study drug.
12. Must not have had an active infection(s) requiring treatment with parenteral anti-infectives within 30 days, or oral anti-infectives within 14 days prior to the first dose of study drug.
13. Must not have confirmed COVID-19. The Baseline visit must be at least 14 days from onset of signs/symptoms or positive SARS-CoV-2 test; symptomatic subjects must have recovered, defined as resolution of fever without use of antipyretics and improvement in symptoms.
14. Must not have suspected COVID-19. Subjects with signs/symptoms suggestive of COVID-19, known exposure, or high-risk behavior should undergo molecular (e.g., PCR) testing to rule out SARS-CoV-2 infection or must be asymptomatic for 14 days from a potential exposure.
15. Must not have a history of an allergic reaction or significant sensitivity to constituents of the study drugs (and its excipients) and/or other products in the same class.
16. Must not have a history of any of the following cardiovascular conditions:
   Recent history (within past 6 months) cerebrovascular accident (CVA), myocardial infarction
   (MI), coronary stenting, coronary bypass surgery.
   Uncontrolled hypertension as defined by a persistent systolic blood pressure (BP) >160 mm Hg or diastolic BP >100 mmHg. For subjects with known hypertension, the subject's BP must be stable for at least 30 days on current, stable anti-hypertensive medications.
   Any other condition which, in the opinion of the Investigator, would put the subject at risk by participating in the protocol.
17. Must not have any clinically relevant or significant ECG abnormalities at Screening, including ECG with QT interval corrected for heart rate (QTc) using Fridericia's correction formula (QTcF) >480 msec. In subjects with ventricular conduction delay (QRS >120 msec), cardiologist consultation is required.
18. For Japan subjects only: must not have positive result of beta-D-glucan or 2 consecutive indeterminate results of beta-D-glucan (screening for Pneumocystis jiroveci infection).

### Prior/Concomitant Medications

1. Must not be using intravenous (IV) or intramuscular (IM) corticosteroids greater than or equal to a 40 mg prednisone-equivalent bolus within 30 days of planned randomization. Must not have been treated with intra-articular, trigger point or tender point, intra-bursa, or intra-tendon sheath corticosteroids in the preceding 8 weeks prior to the first dose of study drug.
2. Subjects must be naïve or have discontinued, if not currently benefitting from, the following
prior to the first dose of study drug per the applicable washout period below or should be at least 5 times the mean terminal elimination half-life of a drug:
   ≥6 months for plasmapheresis
   ≥3 months for belimumab (Benlysta®)
   ≥1 year for rituximab OR ≥6 months if B cells have returned to ≥50 B cells per microliter
   ≥3 months for cyclophosphamide
   ≥4 weeks for abatacept, any anti-tumor necrosis factor (TNF) therapy, and all other biologics
3. Subjects must not have received intravenous immunoglobulin (IVIG) within 30 days prior to the first dose of study drug.
4. Must not have prior exposure for 14 days or more or any prior intolerance to a JAK inhibitor (including but not limited to upadacitinib, tofacitinib, baricitinib, and filgotinib) or a BTK inhibitor. The washout period for JAK and BTK inhibitors prior to the first dose of study drug is ≥30 days or 5 times the half-life, whichever is longer.
5. To avoid combinations of immunosuppressive medications that are not permitted during the study (see criterion 10, above), subjects with prior exposure to the following medications may need to complete a washout period prior to enrollment as specified below or should be at least five times the mean terminal elimination half-life of a drug:
   ≥4 weeks prior to first dose of study drug for MTX, azathioprine, tacrolimus, cyclosporine, mycophenolate.
   ≥8 weeks prior to first dose of study drug for leflunomide if no elimination procedure was followed, or adhere to an elimination procedure (i.e., 11 days with cholestyramine, or 30 days washout with activated charcoal or as per local label).
6. Must not have previous exposure to IFN kinoid vaccination.
7. Must not have prior exposure within 3 months of study Baseline to anti-IFN therapies, including anti-IFN and anti-IFN receptor.
8. Must not have been treated with any investigational drug within 30 days, or 5 half-lives of the drug (whichever is longer), prior to the first dose of study drug or be currently enrolled in another clinical study.
9. Subjects must have discontinued all high-potency opiates at least 1 week and traditional Chinese medicine for at least 30 days prior to the first dose of study drug.
10. Subjects must not have received any live vaccine within 30 days (56 days for Japan) prior to the first dose of study drug.

### Prohibited Medications and Therapy

Medications and therapies with a washout period were prohibited while subjects were on study drug. In addition to the medications listed in the eligibility criteria, the following were prohibited while subjects were on study drug:
Cyclophosphamide
Belimumab
Rituximab
Granulocyte-colony stimulating factor (GCSF)
IVIG
Strong cytochrome P450 3A isoform subfamily (CYP3A) or cytochrome P450 1A2 isoform subfamily (CYP1A2) inhibitors and inducers (examples found in Table 1)
Traditional Chinese medicines
Elective surgery within the first 24 weeks.
High-potency narcotics (unless administered during a hospitalization) including (but not limited to):
   Oxycodone
   Oxymorphone
   Fentanyl
   Levorphanol
   Buprenorphine
   Methadone
   Hydromorphone
   Morphine
   Meperidine
   Of note, low-potency narcotics are permitted to optimize SLE medications.

TABLE 1
Examples of Commonly used Strong CYP3A
or CYP1A2 Inhibitors and Inducers

| Strong CYP3A Inhibitors | Strong CYP3A Inducers |
|---|---|
| Boceprevir | Carbamazepine |
| Cobicistat | Phenytoin |
| Clarithromycin | Rifampin |
| Conivaptan | Rifapentine |
| Grapefruit (fruit or juice) | St. John's Wort |
| Indinavir | |
| Itraconazole | |
| Ketoconazole | |
| Lopinavir/Ritonavir | |
| Mibefradil | |
| Nefazodone | |
| Nelfinavir | |
| Posaconazole | |
| Ritonavir | |
| Saquinavir | |
| Telaprevir | |
| Telithromycin | |
| Troleandomycin | |
| Voriconazole | |
| Strong CYP1A2 Inhibitors Strong | Strong CYP1A2 Inhibitors Strong |
| CYP1A2 Inducers | CYP1A2 Inducers |
| Fluvoxamine | Rifampin |
| Ciprofloxacin | |
| Enoxacin | |
| Zafirlukast | |

### Concomitant Medications/Therapy

Investigators were encouraged to taper steroids in subjects who were stable or improving. The investigator may have decided not to initiate a taper or to delay a taper at any time. Target steroid dose was ≤10 mg QD by Week 16. Steroid dose may have been increased, no more than 5 mg QD per week to a maximum of 25 mg, as needed for rescue purposes up to Week 8. Tapering to the steroid goal of ≤10 mg was permitted up until Week 16. No changes in steroid dose were allowed between Weeks 16 and 24. Starting at Week 24, steroid tapering was again permitted per PI discretion with goal of ≤10 mg QD. Subjects not achieving the steroid goal of ≤10 mg QD at Week 24 were considered non-responders for the primary endpoint. Subjects requiring rescue treatment between Weeks 8 and 48 were considered non-responders from the visit the subject takes rescue treatment. Steroid doses may have been increased or decreased as necessary for safety purposes. Subjects requiring increases of more than 5 mg QD per week were considered non-responders. Subjects requiring an intra-articular injection during the study were considered non-responders. Subjects considered non-responders were allowed to continue on study drug, at the investigator's discretion, if they had not received a prohibited medication.

Subjects must have remained on their background therapy throughout the entirety of the study. The only permitted change of background therapy was steroid tapering. If applicable, subjects continued on their stable doses of nonsteroidal anti-inflammatory drugs (NSAIDs), acetaminophen, and low-potency narcotics. For NSAIDs, acetaminophen/paracetamol, tramadol, codeine, hydrocodone, and propoxyphene that were part of background therapy, changes in dose, including initiation, were not allowed, with the exception of protocol-defined rescue therapy. The following medications taken as needed (PRN) were allowed but were not to be taken within the 24 hours prior to any study visit: NSAIDs, acetaminophen/paracetamol, tramadol, codeine, hydrocodone, and propoxyphene. In the event of tolerability (or other safety) issues, the doses of NSAID and/or acetaminophen may have been decreased or discontinued with substitution of another NSAID.

Any medication or vaccine (including over the counter or prescription medicines, vitamins, and/or herbal supplements) that the subject was receiving at the time of enrollment or received during the study must have been recorded through the post-treatment visit (30-day follow-up phone call). Subjects must have been able to safely discontinue any prohibited medications 30 days prior to initial study drug administration. Subjects must have been consented for the study prior to discontinuing any prohibited medications for the purpose of meeting study eligibility. See Table 2 for a list of allowed concomitant medications.

TABLE 2
Allowed concomitant medications

| Allowed Concomitant | Allowed Concomitant | |
|---|---|---|
| Medications/Therapy | Medications/Therapy | |
| Comments/Notes | Comments/Notes | |
| Methotrexate | ≤20 mg/wk with concomitant | |
| | folic acid ≥5 mg/wk | |
| Azathioprine | ≤150 | mg/day |
| Mycophenolate mofetil | ≤2,000 | mg/day |
| Mycophenolate sodium | ≤1,440 | mg/day |
| Hydroxychloroquine | ≤400 | mg/day |
| Chloroquine | ≤500 | mg/day |
| Quinacrine | ≤100 | mg/day |
| Leflunomide | ≤20 | mg/day |

| | |
|---|---|
| Cyclosporine | Dosed by serum levels |
| Tacrolimus | Dosed by serum levels |
| Corticosteroids | ≤20 mg/day, decrease no faster than 5 mg |
| (prednisone-equivalent) | QD per week. Increase of no more than 5 mg |
| | QD per week regardless of Baseline dose, |
| | maximum dose of 25 mg, through Week 8 |

### Withdrawal of Subjects and Discontinuation of Study

Subjects could request to be discontinued from participating in the study at any time for any reason including but not limited to disease progression or lack of response to treatment. The Investigator could discontinue any subject's study treatment at any time for any reason, including but not limited to disease progression, lack of response to treatment, safety concerns, or failure to comply with the protocol.

At Week 24, the Investigator assessed and documented in source if continuing in the study was in the best interest of the subject. Subjects were discontinued from study drug immediately if any of the following occurred:
Development or worsening of lupus manifestation requiring introduction of certain prohibited medications or dosages when continuation of the study drug would place the subject at risk. The following required discontinuation of study drug immediately:
   Cyclophosphamide
   Belimumab
   Rituximab
   More than one of the following in combination: azathioprine, mycophenolate, leflunomide, MTX, cyclosporine, or tacrolimus
   Plasmapheresis
   High-dose corticosteroids (≥60 mg prednisone equivalent oral or parenteral)
   IVIG
Clinically significant abnormal laboratory results or AEs, which rule out continuation of the study drug, as determined by the investigator
Serious infections (e.g., pneumonia, sepsis) which could not be adequately controlled by anti-infective treatment or put the subject at risk for continued participation in the trial.
The Investigator believed it was in the best interest of the subject.
The subject requested withdrawal from study drug or the study, or the Sponsor decided to discontinue 1 or more treatment groups after the interim analysis.
Subject developed an ECG change considered clinically significant and with reasonable possibility of relationship to study drug or a confirmed absolute QTcF value >500 msec or confirmed increase of ≥60 msec from Baseline.
Subject's QTcF increased from Baseline ≥60 msec, the investigator was to evaluate, confirm the value, and stop treatment.
Subject developed active or latent TB at any time during the study.
The subject became pregnant while on study drug.
Malignancy, except for localized NMSC or CIS of the cervix.
Subject was significantly non-compliant with study procedures, including inconsistent study drug dosing, which put the subject at risk for continued participation in the trial.
Subject developed a GI perforation.
If a diagnosis of deep vein thrombosis, pulmonary embolus, or non-cardiac, non-neurologic arterial thrombosis was confirmed.
Subject developed active neuropsychiatric SLE (excluding lupus headache) as defined by the CNS portion of SLEDAI-2K or BILAG.
Subject developed Class IV lupus nephritis (confirmed by biopsy).

### Study Drug

Upadacitinib or matching placebo manufactured by AbbVie (Table 3) was administered on Day 1 (Baseline) and taken at approximately the same time each day. Subjects were instructed to take study drugs orally, and only 1 tablet of upadacitinib or placebo from the dispensed bottle per day, with or without food. Subjects were instructed to take only 1 capsule or tablet from each dispensed bottle per day. If subjects forgot to take their upadacitinib or matching placebo dose at their regularly scheduled dosing time, they were to take the forgotten dose as soon as they remembered as long as it was at least 10 hours before their next scheduled dose. Otherwise, they were to take the next dose at the next scheduled dosing time. Subject dosing was recorded on a subject dosing diary. The subject was instructed to return all drug containers (even if empty) to the study site personnel at each study visit. Study site personnel documented compliance. Upadacitinib and matching placebo were packaged in bottles with quantities sufficient to accommodate study design. Each kit was labeled per local requirements and this label remained affixed to the kit. Upon receipt, study drug was to be stored as specified on the label and kept in a secure location. Each kit contained a unique kit number. This kit number was assigned to a subject via interactive response technology (IRT) and encoded the appropriate study drug to be dispensed at the subject's corresponding study visit. Site staff completed all blank spaces on the label before dispensing to subjects. Study drug was only to be used for the conduct of the study.

TABLE 3
Study Drug

| | | Investigational |
|---|---|---|
| | Investigational | Product |
| | Product | Placebo |
| Investigational | Upadacitinib | Upadacitinib |
| product name | (ABT-494) | (ABT-494) |
| | | Placebo |
| Active | Upadacitinib | N/A |
| ingredient | (ABT-494) | |
| Mode/Route of | Oral | Oral |
| Administration | | |
| (ROA) | | |
| Dosage Form | Film-Coated | Film-Coated |
| | Tablet | Tablet |
| Dose and units | 30 mg | N/A |
| Frequency of | Daily | Daily |
| administration | | |
| Storage | Room | Room |
| Conditions | temperature | temperature |
| | (15-25° C.) | (15-25° C.) |

N/A = not applicable

### Randomization/Drug Assignment

All subjects were assigned a unique identification number by the IRT at the Screening visit. For subjects who rescreened, the screening number assigned by the IRT at the initial Screening visit was used. The IRT assigned a randomization number that encoded the subject's treatment group assignment according to the randomization schedule generated by the statistics department at AbbVie. Randomization was stratified based on the following factors:
Baseline corticosteroid dose above 10 mg prednisone-equivalent (≤10 mg or >10 mg)
Screening SLEDAI-2K (<10 or ≥10).
High vs low vs not applicable IFN score (approximately 80% high)
This stratification factor did not apply to subjects in China.

Baseline immunosuppressant (azathioprine, tacrolimus, cyclosporine, MTX, mycophenolate, or leflunomide) (yes/no)

Once approximately 2000 of total subjects had been randomized who were IFN signature low, further enrollment of such subjects may have been suspended. The investigator, study site personnel, and the subject remained blinded to each subject's treatment throughout the study. To maintain the blind, upadacitinib tablets and matching placebo tablets provided for the study were identical in appearance.

Results from Week 24 Interim Analysis

### Demographics

A table summarizing the demographics for subjects in the placebo and upadacitinib 30 mg QD treatment groups of the clinical study are provided in Table 4.

TABLE 4
Demographic characteristics of study groups.

| Demographic | Group | | | |
|---|---|---|---|---|
| Characteristics, | PBO | | UPA 30 mg | |
| mean (SD) or n (%) | (N = 75) | | (N = 62) | |
| Sex | | | | |
| Female | 75 | (100) | 57 | (91.9) |
| Age (years) | 41.7 | (12.05) | 42.5 | (11.89) |
| Weight (kg) | 70.06 | (21.142) | 73.06 | (21.919) |
| Body Mass Index (kg/M\hat{ }2) | 27.20 | (7.472) | 27.44 | (7.365) |
| Race | | | | |
| White | 43 | (57.3) | 34 | (54.8) |
| Black or African American | 4 | (5.3) | 9 | (14.5) |
| American Indian/Alaska | 3 | (4.0) | 0 | |
| Native | | | | |
| Native Hawaiian/Other | 0 | | 0 | |
| Pacific Islander | | | | |
| Asian | 23 | (30.7) | 13 | (21.0) |
| Other | 0 | | 0 | |
| Multiple | 2 | (2.7) | 6 | (9.7) |
| Region | | | | |
| North America | 23 | (30.7) | 26 | (41.9) |
| South/Central America | 15 | (20.0) | 11 | (17.7) |
| Western Europe | 4 | (5.3) | 9 | (14.5) |
| Eastern Europe | 7 | (9.3) | 3 | (4.8) |
| Asia Japan | 9 | (12.0) | 4 | (6.5) |
| Asia Other | 14 | (18.7) | 6 | (9.7) |
| Other | 3 | (4.0) | 3 | (4.8) |

A table of baseline disease characteristics for subjects in the placebo and upadacitinib 30 mg QD treatment groups of the clinical study are provided in Table 5.

TABLE 5
Baseline disease characteristics of study groups.
Baseline Disease Characteristics

| | |
|---|---|
| Baseline Disease | Group |

| Characteristics, | PBO | UPA 30 mg |
|---|---|---|
| mean (SD) or n (%) | (N = 75) | (N = 62) |
| SLEDAI-2K Score | 9.0 (3.91) | 9.0 (2.77) |
| Patient's global | (N = 73) | (N = 60) |
| assessment of disease | 49.1 (27.21) | 50.4 (23.72) |
| activity score | | |
| Physician's global | 1.75 (0.440) | 1.70 (0.438) |
| assessment of disease | | |
| activity score | | |
| SF-36 physical | (N = 74) | (N = 60) |
| component summary | 40.3 (10.10) | 38.2 (9.12) |
| score | | |
| SF-36 mental | (N = 74) | (N = 60) |
| component summary | 42.4 (12.11) | 43.7 (12.27) |
| score | | |
| Anti-dsDNA status | | |
| Positive | 33 (44.0) | 28 (45.2) |
| Negative | 42 (56.0) | 34 (54.8) |
| SDAI2K-Proteinuria | | |
| No | 0 | 0 |
| Yes | 75 (100) | 62 (100) |
| SDAI2K-Arthritis | | |
| No | 7 (9.3) | 0 |
| Yes | 68 (90.7) | 62 (100) |
| SDAI2K-Rash | | |
| No | 32 (42.7) | 27 (43.5) |
| Yes | 43 (57.3) | 35 (56.5) |
| SDAI2K-Low Complement | | |
| No | 42 (56.0) | 30 (48.4) |
| Yes | 33 (44.0) | 32 (51.6) |
| C3 status (<90.0 mg/dL) | | |
| No | 41 (54.7) | 38 (61.3) |
| Yes | 34 (45.3) | 24 (38.7) |
| C4 status (<10.0 mg/dL) | | |
| No | 54 (72.0) | 50 (80.6) |
| Yes | 21 (28.0) | 12 (19.4) |
| Corticosteroid use | | |
| No | 19 (25.3) | 20 (32.3) |
| Yes | 56 (74.7) | 42 (67.7) |
| Immunosuppressant Use | | |
| No | 47 (62.7) | 37 (59.7) |
| Yes | 28 (37.3) | 25 (40.3) |

A summary of subject disposition in the placebo and upadacitinib 30 mg QD treatment groups of the clinical study is provided in Table 6.

TABLE 6
Subject disposition

Group

| Subject Disposition, n | PBO | UPA 30 mg |
|---|---|---|
| (%) | (N = 75) | (N = 62) |
| Randomized | 75 (100) | 62 (100) |
| Completed the Study | 27 (36.0) | 24 (38.7) |
| Withdrawal from the | 17 (22.7) | 11 (17.7) |
| Study (Primary | | |
| Reasons) | | |
| Adverse event | 2 (2.7) | 3 (4.8) |
| Withdrawal by | 6 (8.0) | 5 (8.1) |
| subject | | |
| Lost to follow-up | 2 (2.7) | 2 (3.2) |
| COVID-19 infection | 0 | 0 |
| COVID-19 | 0 | 0 |
| logistical restrictions | | |
| Other | 7 (9.3) | 1 (1.6) |
| Sponsor Decision | 0 | 0 |
| Based on Interim | | |
| Analysis | | |
| Withdrawal from the | 17 (22.7) | 11 (17.7) |
| Study (All Reasons) | | |
| Adverse event | 4 (5.3) | 4 (6.5) |
| Withdrawal by | 7 (9.3) | 5 (8.1) |
| subject | | |
| Lost to follow-up | 2 (2.7) | 2 (3.2) |
| COVID-19 infection | 0 | 0 |
| COVID-19 | 0 | 0 |
| logistical restrictions | | |
| Other | 7 (9.3) | 2 (3.2) |
| Sponsor Decision | 0 | 0 |
| Based on Interim | | |
| Analysis | | |
| Completed 24 Weeks | 66 (88.0) | 56 (90.3) |
| Study Period | | |
| Completed the Study | 31 (41.3) | 25 (40.3) |
| Drug | | |
| Discontinued from | | |
| the Study Drug | | |
| (Primary Reasons) | | |
| Adverse event | 5 (6.7) | 5 (8.1) |
| Withdrew consent | 3 (4.0) | 6 (9.7) |
| Lost to follow-up | 2 (2.7) | 2 (3.2) |
| Lack of efficacy | 4 (5.3) | 0 |
| COVID-19 infection | 0 | 0 |
| COVID-19 logistical | 0 | 0 |
| restrictions | | |
| Other | 5 (6.7) | 0 |
| Sponsor Decision | 0 | 0 |
| Based on Interim | | |
| Analysis | | |
| Discontinued from | 19 (25.3) | 13 (21.0) |
| the Study Drug (All | | |
| Reasons) | | |
| Adverse event | 5 (6.7) | 6 (9.7) |
| Withdrew consent | 4 (5.3) | 6 (9.7) |
| Lost to follow-up | 2 (2.7) | 2 (3.2) |
| Lack of efficacy | 4 (5.3) | 1 (1.6) |
| COVID-19 infection | 0 | 0 |
| COVID-19 logistical | 0 | 0 |
| restrictions | | |
| Other | 5 (6.7) | 1 (1.6) |
| Sponsor Decision | 0 | 0 |
| Based on Interim | | |
| Analysis | | |

### Efficacy

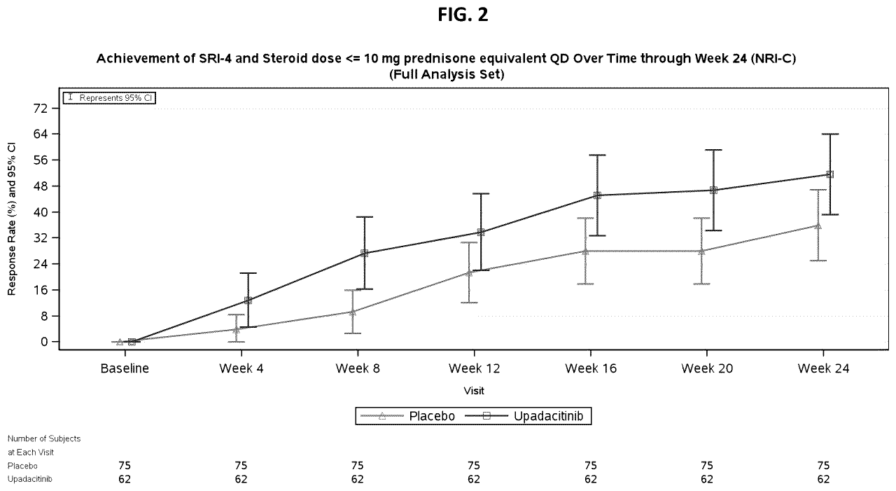

A graphical depiction of response rate over time with respect to the primary endpoint (achievement of SRI-4 and Steroid Dose ≤10 mg prednisone equivalent QD) for subjects treated with placebo and upadacitinib 30 mg QD over 24 weeks is provided as FIG. 2. Upadacitinib 30 mg showed higher efficacy than placebo (51.6% vs. 36.0%; stratum-adjusted treatment difference: 14.0%; p=0.067).

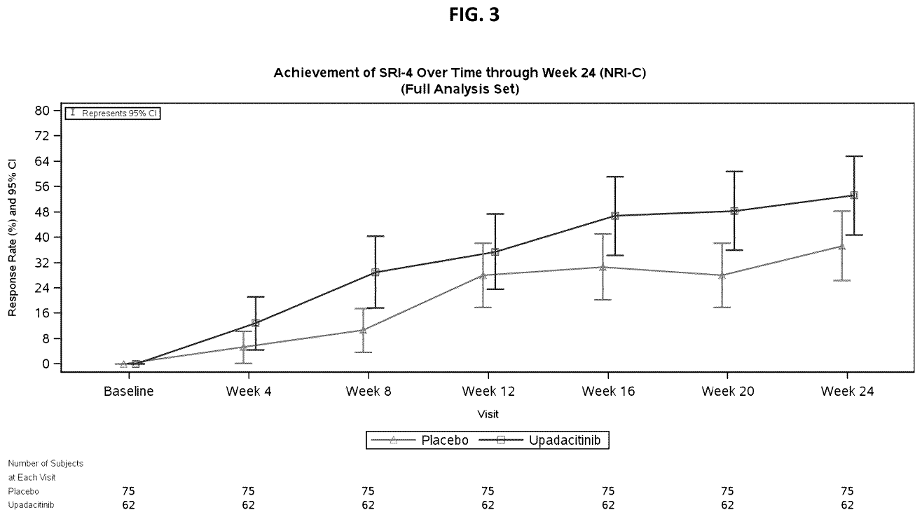

A graphical depiction of response rate overtime with respect to achievement of SRI-4 for subjects treated with placebo and upadacitinib 30 mg QD over 24 weeks is provided as FIG. 3. Upadacitinib 30 mg showed higher efficacy than placebo in SRI-4 at Week 24 (53.6% vs. 37.3% with p=0.046).

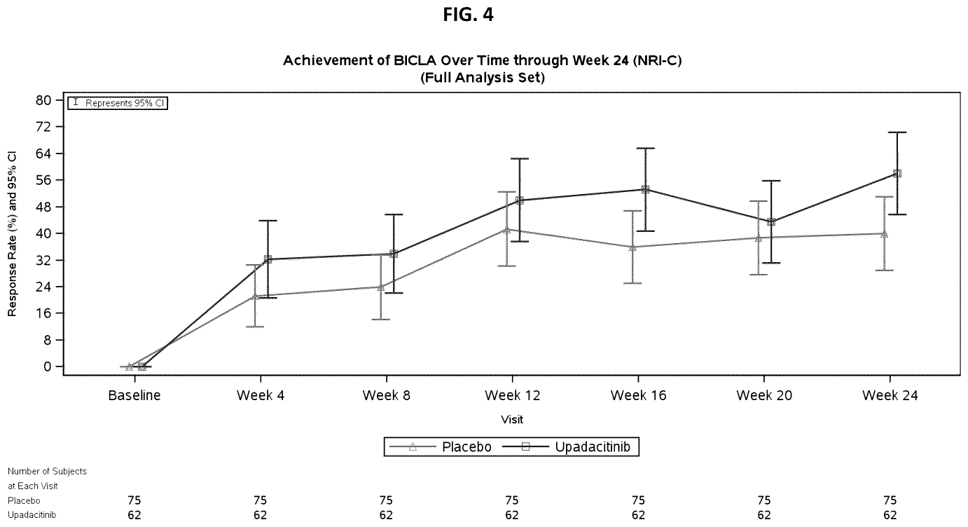

A graphical depiction of response rate over time with respect to achievement of BICLA for subjects treated with placebo and upadacitinib 30 mg QD over 24 weeks is provided as FIG. 4. Upadacitinib 30 mg showed higher efficacy than placebo in BICLA at Week 24 (58.1% vs. 40.0% with p=0.056).

A tabular summary of the primary and secondary endpoints and associated statistics for the full analysis set are provided in Table 7, which further shows that upadacitinib 30 mg achieved greater efficacy than placebo in LLDAS at Week 24 (p<0.001), as well as a lower flare rate through Week 24 (p=0.075).

TABLE 7
Summary of results for primary and secondary endpoints among full analysis set

| | | |
|---|---|---|
| | PBO | UPA 30 mg |
| Endpoint | (N = 75) | (N = 62) |

Primary Endpoint

| | | | | |
|---|---|---|---|---|
| SRI-4 and Prednisone Equivalent | 27 | (36.0) | 32 | (51.6) |
| Steroid Dose ≤10 mg at Week 24; n | | | | |
| (%) | | | | |

| | |
|---|---|
| Diff (−PBO) [95% CI], p-value | 14.0 [−1.0, 29.1], |
| | p = 0.067 |

Secondary Endpoints

| SRI-4 at Week 24; n (%) | 28 | (37.3) | 33 | (53.2) |
|---|---|---|---|---|
| Diff (−PBO) [95% CI], p-value | | | 15.3 [0.2, 30.3], | |
| | | | p = 0.046 | |
| BICLA at Week 24; n (%) | 30 | (40.0) | 36 | (58.1) |
| Diff (−PBO) [95% CI], p-value | | | 15.6 [−0.4, 31.6], | |
| | | | p = 0.056 | |
| LLDAS at Week 24; n (%) | 10 | (13.3) | 27 | (43.5) |
| Diff (−PBO) [95% CI], p-value | | | 29.5 [16.4, 42.5], | |
| | | | p < 0.001 | |
| Prednisone Equivalent Steroid Dose | (N = 56) | | (N = 48) | |
| at Week 24; LS Mean Change (SE) | −0.66 (0.461) | | −0.64 (0.486) | |
| Diff (−PBO) [95% CI], p-value | | | 0.03 [−1.02, 1.08], | |
| | | | p = 0.961 | |
| Flares per SELENA SLEDAI Flare | | | | |
| Index through Week 24 | | | | |
| Mild/Moderate; Events/PY [95% CI] | 2.01 | [1.53, 2.49] | 1.66 | [1.19, 2.13] |
| Diff (−PBO) [95% CI], p-value | | | −0.35 [−1.03, 0.32], | |
| | | | p = 0.304 | |
| Severe; Events/PY [95% CI] | 0.36 | [0.16, 0.56] | 0.07 | [−0.03, 0.17] |
| Diff (−PBO) [95% CI], p-value | | | −0.29 [−0.52, −0.07], | |
| | | | p = 0.011 | |
| Any flares; Events/PY [95% CI] | 2.37 | [1.85, 2.90] | 1.73 | [1.25, 2.21] |

| | |
|---|---|
| Diff (−PBO) [95% CI], p-value | −0.64 [−1.35, 0.07], |
| | p = 0.075 |

CI: Confidence interval.
P-values for treatment difference were based on CMH test for categorical endpoints and MMRM for continuous endpoints, controlling for stratification factors and based on normal approximation for flare rate through Week 24.

### Pharmacokinetics

The model-estimated median (9000 prediction interval) steady-state (SS) C_{avg} in subjects with SLE was 28.4 (19.1-46.6) ng/mL for upadacitinib 30 mg QD.

### Exposure-Response for Efficacy Endpoints

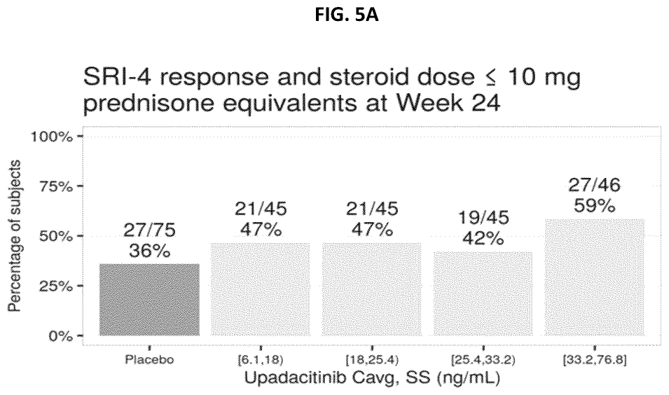

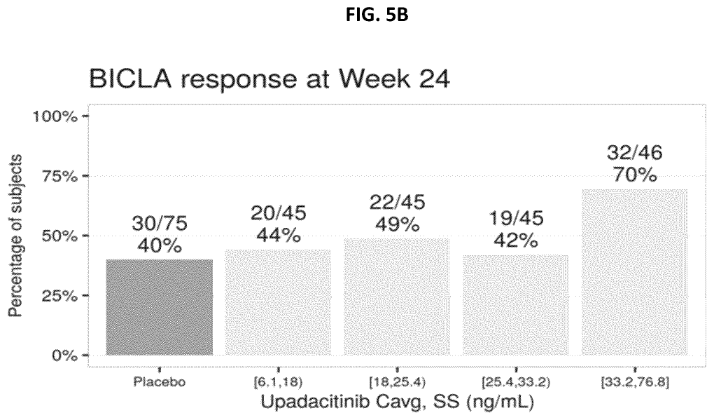

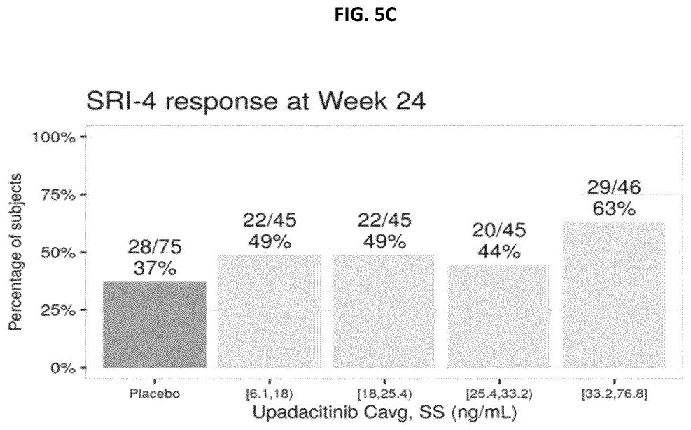

Quartile plots for the relationships between upadacitinib steady-state (SS) C_{avg} and efficacy responses at Week 24 are shown in FIG. 5A to 5C. Specifically, FIG. 5A is an exposure-response quartile plot for upadacitinib efficacy at week 24 for the efficacy endpoint of SLE Responder Index (SRI-4) and Steroid Dose ≤10 mg prednisone equivalent QD, FIG. 5B is an exposure-response quartile plot for upadacitinib efficacy at week 24 for the efficacy endpoint of BILAG-Based Combined Lupus Assessment (BICLA), and FIG. 5C is an exposure-response quartile plot for upadacitinib efficacy at week 24 for the efficacy endpoint of SLE Responder Index (SRI-4). Based on logistic regression analyses, upadacitinib plasma exposures had a statistically significant relationship (p<0.05) with SRI-4 and steroid dose ≤10 mg prednisone equivalent, SRI-4, and BICLA at Week 24.

A plateau for efficacy was not reached at the upadacitinib plasma exposures evaluated in the study (associated with 30 mg QD).

The highest upadacitinib steady-state (SS) C_{avg} quartile (which would be equivalent to 45 mg QD exposures) was consistently associated with the highest observed response across the three endpoints and was the primary driver for upadacitinib exposure-response trends. The exposure-response model-predicted median (90% prediction interval) percentage of subjects achieving SRI-4 and steroid dose ≤10 mg prednisone equivalent QD, BICLA, and SRI-4 at Week 24 is 50% (42% to 58%), 57% (50% to 65%), and 55% (47% to 62%), respectively, for upadacitinib 30 mg UPA QD compared to 38% (31% to 46%), 39% (32% to 46%), and 40% (32% to 47%), respectively, for placebo. A upadacitinib 45 mg QD dose is predicted to provide 6% to 8% greater responses compared to 30 mg QD.

### Biomarker Analysis

Upadacitinib 30 mg treatment group showed a significant decrease in the levels of C3, C4 complement, and IgM, compared to Baseline, as well as significant decreases in the level of anti-dsDNA autoantibodies and IgG. Increased circulating memory, naïve, and total B cells were also observed.

### Example 2: Safety and Efficacy of Upadacitinib in Systemic Lupus Erythematosus (SLE)

This was an extension of the Phase 2 Study of Example 1. Specifically, the study of Example 1 was extended a further 24 weeks to a total of 48 weeks to further assess the maintenance of efficacy and safety of upadacitinib 30 mg treatment beyond 24 weeks. A schematic illustration of the study is shown in FIG. 1. In total, the study included 62 subjects in the upadacitinib 30 mg group and 75 subjects in the Placebo group.

### Results

### Efficacy

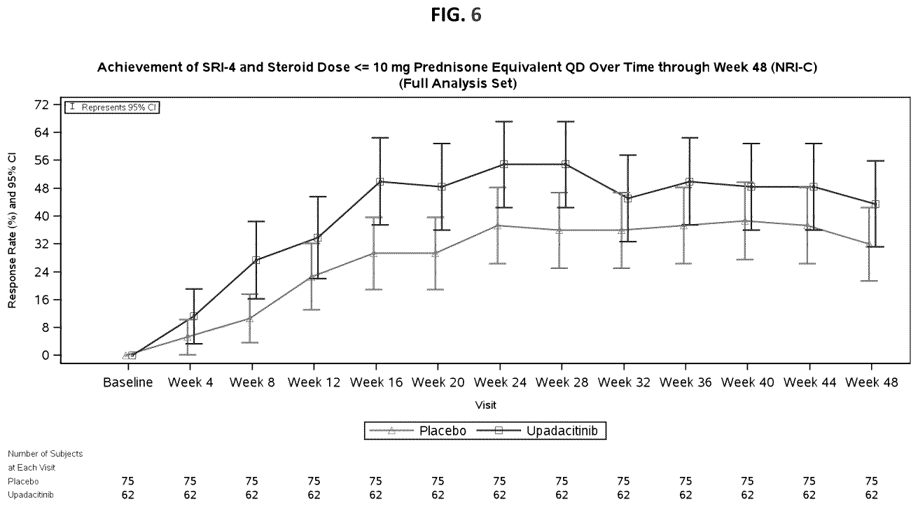

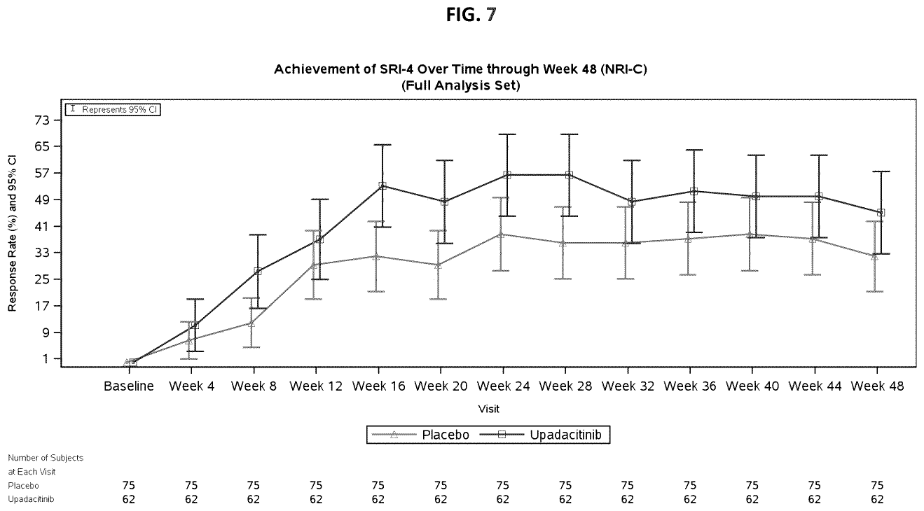

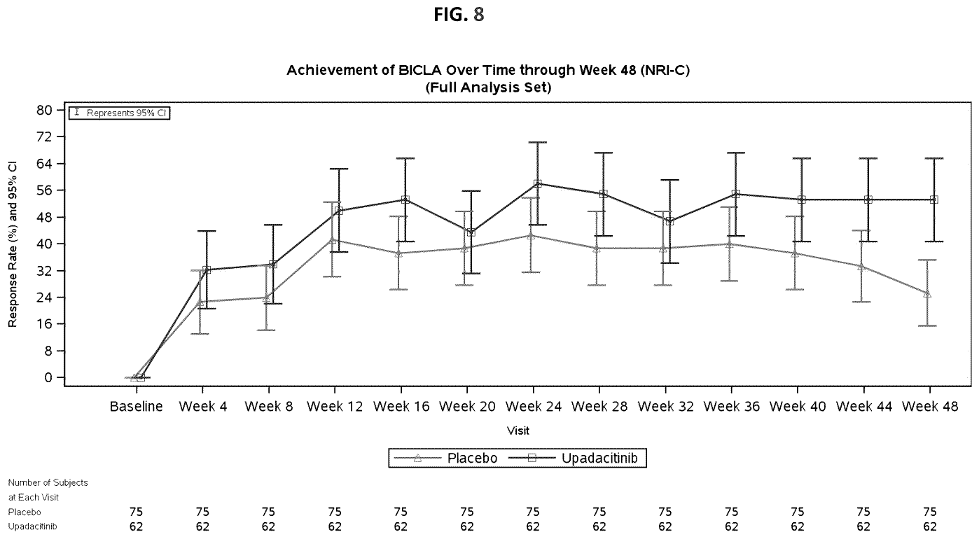

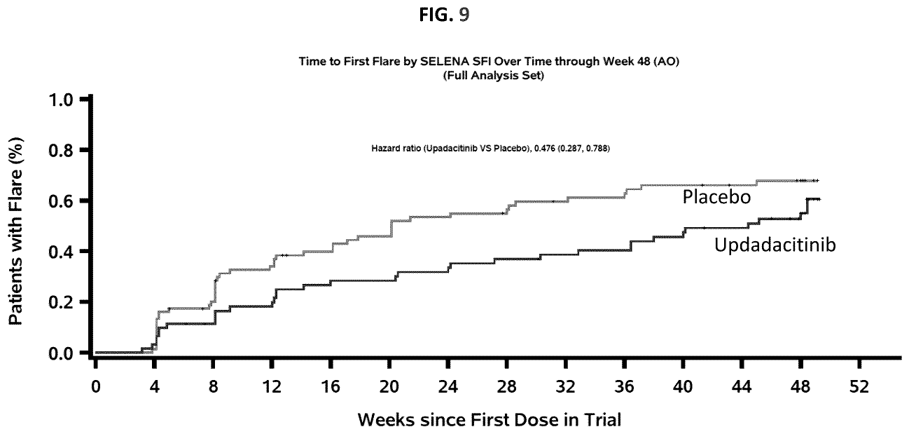

The Week 24 and Week 48 results from selected efficacy endpoints for continued groups are presented in Table 8 and FIGS. 6-9. Of note, Week 24 results in Table 8 are based on the final database; therefore, they are slightly different from the 24-week values in corresponding Table 7 of Example 1.

After Week 24, the response rates in BICLA and SRI-4 in both the upadacitinib 30 mg group were generally maintained, while the placebo rate either remained stable (SRI-4) or decreased (BICLA). Upadacitinib 30 mg showed a higher response rate than Placebo (53.200 vs. 25.30; stratum-adjusted treatment difference: 30.8; p 0.001) for achievement of BICLA at Week 48. Upadacitinib 30 mg showed a higher response rate than Placebo (45.2% vs. 32.0%; stratum-adjusted treatment difference: 30.4; p=0.075) for achievement of SRI-4 at Week 48. Upadacitinib 30 mg also showed reductions in flare and delays in time-to-first flare compared to placebo. Overall, the totality of the data suggests that upadacitinib 30 mg QD provides additional benefits over that of background medication alone in subjects with moderate to severe SLE.

TABLE 8
Efficacy Results at Week 24 and Week 48 (Full Analysis Set; Continued Groups)

| | | |
|---|---|---|
| | PBO | UPA 30 mg |
| Endpoint | (N = 75) | (N = 62) |

SRI-4 and Prednisone Equivalent Steroid Dose ≤10 mg

| Week 24; n (%) | 28 | (37.3) | 34 | (54.8) |
|---|---|---|---|---|
| Adj. Diff¹ (−PBO) [95% CI], | | | 16.9 [1.8, 31.9], | |
| p-value | | | p = 0.028 | |
| Week 48; n (%) | 24 | (32.0) | 27 | (43.5) |

| | |
|---|---|
| Adj. Diff¹ (−PBO) [95% CI], | 12.0 [−2.7, 26.7], |
| p-value | p = 0.109 |

SRI-4

| Week 24; n (%) | 29 | (38.7) | 35 | (56.5) |
|---|---|---|---|---|
| Adj. Diff¹ (−PBO) [95% CI], | | | 18.1 [3.0, 33.2], | |
| p-value | | | p = 0.018 | |
| Week 48; n (%) | 24 | (32.0) | 28 | (45.2) |

| | |
|---|---|
| Adj. Diff¹ (−PBO) [95% CI], | 13.4 [−1.4, 28.2], |
| p-value | p = 0.075 |

BICLA

| Week 24; n (%) | 32 | (42.7) | 36 | (58.1) |
|---|---|---|---|---|
| Adj. Diff¹ (−PBO) [95% CI], | | | 13.9 [−2.2, 30.1], | |
| p-value | | | p = 0.091 | |
| Week 48; n (%) | 19 | (25.3) | 33 | (53.2) |

| | |
|---|---|
| Adj. Diff¹ (−PBO) [95% CI], | 30.8 [17.6, 44.1], |
| p-value | p < 0.001 |

LLDAS

| Week 24; n (%) | 10 | (13.3) | 28 | (45.2) |
|---|---|---|---|---|
| Adj. Diff¹ (−PBO) [95% CI], | | | 31.0 [18.1, 44.0], | |
| p-value | | | p < 0.001 | |
| Week 48; n (%) | 18 | (24.0) | 31 | (50.0) |

| | |
|---|---|
| Adj. Diff¹ (−PBO) [95% CI], | 26.6 [12.9, 40.4], |
| p-value | p < 0.001 |

Prednisone Equivalent Steroid Dose

| | | |
|---|---|---|
| Week 24; LS Mean Change (SE) | (N = 56) | (N = 48) |
| | −0.65 (0.471) | −0.62 (0.496) |
| Adj. Diff¹ (−PBO) [95% CI], | | 0.03 [−1.05, 1.10], |
| p-value | | p = 0.963 |
| Week 48; LS Mean Change (SE) | (N = 40) | (N = 38) |
| | −1.49 (0.503) | −1.20 (0.524) |
| Adj. Diff¹ (−PBO) [95% CI], | | 0.28 [−0.90, 1.46], |
| p-value | | p = 0.636 |

Flares per SELENA SLEDAI Flare Index

| Through Week 24 | | | | |
|---|---|---|---|---|
| Mild/Moderate; Events/PY | 2.45 | [1.92, 2.99] | 1.76 | [1.28, 2.25] |
| [95% CI] | | | | |
| Adj. Diff¹ (−PBO) [95% | | | −0.69 [−1.41, 0.03], | |
| CI], p-value | | | p = 0.059 | |
| Severe; Events/PY [95% CI] | 0.36 | [0.16, 0.56] | 0.10 | [−0.01, 0.22] |
| Adj. Diff¹ (−PBO) [95% | | | −0.26 [−0.49, −0.02], | |
| CI], p-value | | | p = 0.033 | |
| Any flares; Events/PY [95% | 2.81 | [2.24, 3.38] | 1.87 | [1.37, 2.36] |
| CI] | | | | |
| Adj. Diff¹ (−PBO) [95% CI], | | | −0.95 [−1.70, −0.19], | |
| p-value | | | p = 0.014 | |
| Through Week 48 | | | | |
| Mild/Moderate; Events/PY | 2.50 | [2.09, 2.90] | 1.88 | [1.50, 2.26] |
| [95% CI] | | | | |
| Adj. Diff¹ (−PBO) [95% | | | −0.62 [−1.17, −0.06], | |
| CI], p-value | | | p = 0.029 | |
| Severe; Events/PY [95% CI] | 0.33 | [0.18, 0.47] | 0.16 | [0.05, 0.27] |
| Adj. Diff¹ (−PBO) [95% | | | −0.17 [−0.35, 0.02], | |
| CI], p-value | | | p = 0.074 | |
| Any flares; Events/PY [95% | 2.82 | [2.39, 3.25] | 2.04 | [1.64, 2.43] |
| CI] | | | | |
| Adj. Diff¹ (−PBO) [95% | | | −0.78 [−1.37, −0.20], | |
| CI], p-value | | | p = 0.008 | |
| 50% Reduction of Tender or | | | | |
| Swollen Lupus Joints among | | | | |
| Subjects with Total >=6 Affected | | | | |
| Joints | | | | |
| Week 24; n (%) | 38/59 | (64.4) | 40/59 | (67.8) |
| Adj. Diff¹ (−PBO) [95% | | | 4.9 [−10.4, 20.1], | |
| CI], p-value | | | p = 0.532 | |
| Week 48; n (%) | 26/59 | (44.1) | 34/59 | (57.6) |

| | |
|---|---|
| Adj. Diff¹ (−PBO) [95% | 14.6 [−1.1, 30.4], |
| CI], p-value | p = 0.069 |

CI: Confidence interval.
¹Adj. Diff: stratum-adjusted treatment difference except for the flares, which were summarized as observed.
P-values for treatment difference were based on the CMH test for categorical endpoints and MMRM for continuous endpoints, controlling for stratification factors and based on the normal approximation for flare rate through Week 24 or through Week 48.

### Exposure-Response for Efficacy Endpoints

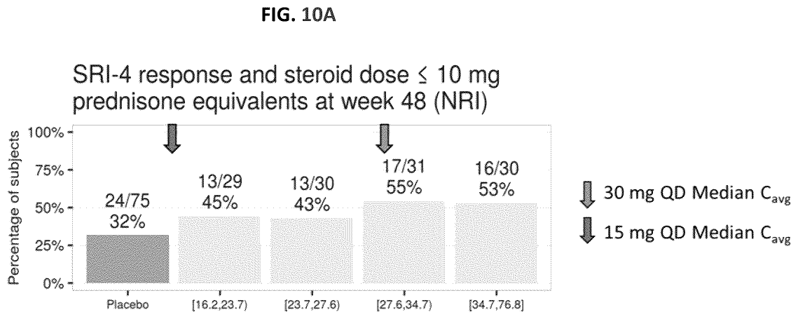

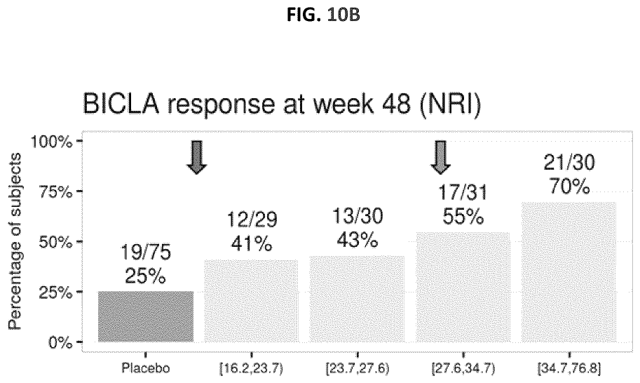

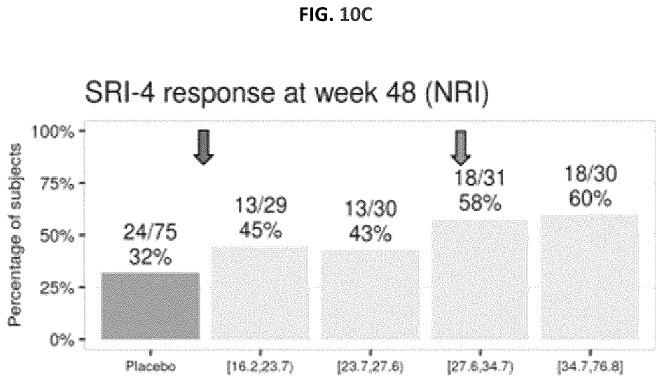

There was a clear exposure-response relationship (p<0.05) for upadacitinib with SRI-4 and steroid dose ≤10 mg prednisone equivalent, SRI-4, and BICLA at Week 48. Quartile plots for the relationships between upadacitinib C_{avg} and efficacy responses at Week 48 are shown in FIGS. 10A-10C (SRI-4 and steroid dose ≤10 mg prednisone equivalent, SRI-4, and BICLA, respectively). Based on logistic regression analysis, there is an exposure-response relationship (p <0.05) for upadacitinib with each of the Week 48 efficacy endpoints (SRI-4 and steroid dose (10 mg prednisone equivalent QD, BICLA, and SRI-4).

The exposure-response model-predicted median (90% prediction interval) percentage of subjects achieving SRI-4 and steroid dose ≤10 mg prednisone equivalent QD, BICLA, and SRI-4 at Week 48 is 50% (41% to 58%), 56% (47% to 64%), and 54% (45% to 61%), respectively, for upadacitinib 30 mg UPA QD compared to 34% (25% to 44%), 25% (18% to 34%), and 32% (24% to 42%), respectively, for placebo. A upadacitinib 30 mg QD dose is predicted to provide 8% to 16% greater efficacy responses compared to 15 mg QD.

### Biomarkers

The Week 48 predefined biomarker analyses confirmed the trends observed at Week 24. Namely, treatment with upadacitinib 30 mg QD resulted in meaningful decreases in anti-dsDNA autoantibodies and an early reduction (a known effect of JAK inhibitors) followed by a progressive normalization in complement C3 and C4 levels.

What is claimed is:

1. A method of treating a human patient having moderately to severely active systemic lupus erythematosus, the method comprising orally administering once daily to the patient 30 mg of upadacitinib.

2. A method of treating a human patient having moderately to severely active systemic lupus erythematosus, the method comprising orally administering once daily to the patient 15 mg of upadacitinib.

3. A method of treating a human patient having moderately to severely active systemic lupus erythematosus, the method comprising orally administering once daily to the patient 45 mg of upadacitinib.

4. The method of claim 3, wherein the 45 mg of upadacitinib is an induction dose, followed by orally administering once daily to the patient a maintenance dose.

5. The method of claim 4, wherein the maintenance dose is 30 mg.

6. The method of claim 4, wherein the maintenance dose is 15 mg.

7. The method of any one of claims 1-6, wherein the method results in a SLE Responder Index (SRI)-4 response and Prednisone Equivalent Steroid Dose ≤10 mg at 52 weeks after the first daily administration.

8. The method of any one of claims 1-6, wherein the method results in a SLE Responder Index (SRI)-4 response at 52 weeks after the first daily administration.

9. The method of any one of claims 1-6, wherein the method results in a BILAG-Based Combined Lupus Assessment (BICLA) response at 52 weeks after the first daily administration.

10. The method of any one of claims 1-6, wherein the method results in a Lupus Low Disease Activity State (LLDAS) response at 52 weeks after the first daily administration.

11. The method of any one of claims 1-6, wherein the method results in a reduction in steroid burden, assessed as change from Baseline in prednisone equivalent steroid dose at Week 52.

12. The method of any one of claims 1-6, wherein the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 52.

13. The method of any one of claims 1-6, wherein the method results in a SLE Responder Index (SRI)-4 response and Prednisone Equivalent Steroid Dose ≤10 mg at 48 weeks after the first daily administration.

14. The method of any one of claims 1-6, wherein the method results in a SLE Responder Index (SRI)-4 response at 48 weeks after the first daily administration.

15. The method of any one of claims 1-6, wherein the method results in a BILAG-Based Combined Lupus Assessment (BICLA) response at 48 weeks after the first daily administration.

16. The method of any one of claims 1-6, wherein the method results in a Lupus Low Disease Activity State (LLDAS) response at 48 weeks after the first daily administration.

17. The method of any one of claims 1-6, wherein the method results in a reduction in steroid burden, assessed as change from Baseline in prednisone equivalent steroid dose at Week 48.

18. The method of any one of claims 1-6, wherein the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 48.

19. The method of any one of claims 1-6, wherein the method results in a SLE Responder Index (SRI)-4 response and Prednisone Equivalent Steroid Dose 10 mg at 24 weeks after the first daily administration.

20. The method of any one of claims 1-6, wherein the method results in a SLE Responder Index (SRI)-4 response at 24 weeks after the first daily administration.

21. The method of any one of claims 1-6, wherein the method results in a BILAG-Based Combined Lupus Assessment (BICLA) response at 24 weeks after the first daily administration.

22. The method of any one of claims 1-6, wherein the method results in a Lupus Low Disease Activity State (LLDAS) response at 24 weeks after the first daily administration.

23. The method of any one of claims 1-6, wherein the method results in a reduction in steroid burden, assessed as change from Baseline in prednisone equivalent steroid dose at Week 24.

24. The method of any one of claims 1-6, wherein the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 24.

25. The method of any one of claims 1-6, wherein the method results in a SLE Responder Index (SRI)-4 response and Prednisone Equivalent Steroid Dose ≤10 mg at 20 weeks after the first daily administration.

26. The method of any one of claims 1-6, wherein the method results in a SLE Responder Index (SRI)-4 response at 20 weeks after the first daily administration.

27. The method of any one of claims 1-6, wherein the method results in a BILAG-Based Combined Lupus Assessment (BICLA) response at 20 weeks after the first daily administration.

28. The method of any one of claims 1-6, wherein the method results in a Lupus Low Disease Activity State (LLDAS) response at 20 weeks after the first daily administration.

29. The method of any one of claims 1-6, wherein the method results in a reduction in steroid burden, assessed as change from Baseline in prednisone equivalent steroid dose at Week 20.

30. The method of any one of claims 1-6, wherein the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 20.

31. The method of any one of claims 1-6, wherein the method results in a SLE Responder Index (SRI)-4 response and Prednisone Equivalent Steroid Dose 10 mg at 16 weeks after the first daily administration.

32. The method of any one of claims 1-6, wherein the method results in a SLE Responder Index (SRI)-4 response at 16 weeks after the first daily administration.

33. The method of any one of claims 1-6, wherein the method results in a BILAG-Based Combined Lupus Assessment (BICLA) response at 16 weeks after the first daily administration.

34. The method of any one of claims 1-6, wherein the method results in a Lupus Low Disease Activity State (LLDAS) response at 16 weeks after the first daily administration.

35. The method of any one of claims 1-6, wherein the method results in a reduction in steroid burden, assessed as change from Baseline in prednisone equivalent steroid dose at Week 16.

36. The method of any one of claims 1-6, wherein the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 16.

37. The method of any one of claims 1-6, wherein the method results in a SLE Responder Index (SRI)-4 response and Prednisone Equivalent Steroid Dose ≤10 mg at 12 weeks after the first daily administration.

38. The method of any one of claims 1-6, wherein the method results in a SLE Responder Index (SRI)-4 response at 12 weeks after the first daily administration.

39. The method of any one of claims 1-6, wherein the method results in a BILAG-Based Combined Lupus Assessment (BICLA) response at 12 weeks after the first daily administration.

40. The method of any one of claims 1-6, wherein the method results in a Lupus Low Disease Activity State (LLDAS) response at 12 weeks after the first daily administration.

41. The method of any one of claims 1-6, wherein the method results in a reduction in steroid burden, assessed as change from Baseline in prednisone equivalent steroid dose at Week 12.

42. The method of any one of claims 1-6, wherein the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 12.

43. The method of any one of claims 1-6, wherein the method results in a SLE Responder Index (SRI)-4 response and Prednisone Equivalent Steroid Dose ≤10 mg at 8 weeks after the first daily administration.

44. The method of any one of claims 1-6, wherein the method results in a SLE Responder Index (SRI)-4 response at 8 weeks after the first daily administration.

45. The method of any one of claims 1-6, wherein the method results in a BILAG-Based Combined Lupus Assessment (BICLA) response at 8 weeks after the first daily administration.

46. The method of any one of claims 1-6, wherein the method results in a Lupus Low Disease Activity State (LLDAS) response at 8 weeks after the first daily administration.

47. The method of any one of claims 1-6, wherein the method results in a reduction in steroid burden, assessed as change from Baseline in prednisone equivalent steroid dose at Week 8.

48. The method of any one of claims 1-6, wherein the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 8.

49. The method of any one of claims 1-6, wherein the method results in a SLE Responder Index (SRI)-4 response and Prednisone Equivalent Steroid Dose 10 mg at 4 weeks after the first daily administration.

50. The method of any one of claims 1-6, wherein the method results in a SLE Responder Index (SRI)-4 response at 4 weeks after the first daily administration.

51. The method of any one of claims 1-6, wherein the method results in a BILAG-Based Combined Lupus Assessment (BICLA) response at 4 weeks after the first daily administration.

52. The method of any one of claims 1-6, wherein the method results in a Lupus Low Disease Activity State (LLDAS) response at 4 weeks after the first daily administration.

53. The method of any one of claims 1-6, wherein the method results in a reduction in steroid burden, assessed as change from Baseline in prednisone equivalent steroid dose at Week 4.

54. The method of any one of claims 1-6, wherein the method results in a reduction in the number of mild, moderate, or severe flares per patient-year (respectively and overall) by Safety of Estrogens in Lupus Erythematosus National Assessment (SELENA) SLEDAI Flare Index (SFI), assessed by number and types of flare (mild/moderate, severe, and any) per subject through Week 4.

55. The method of any one of claims 1-54, wherein the method results in a significant decrease in the levels of anti-dsDNA autoantibodies and complement C3 and C4.

56. The method of any one of claims 1-55, wherein the method results in a significant increase in the numbers of circulating memory, naïve, and total B cells.

57. The method of any one of claims 1-56, wherein the patient has had an inadequate response or intolerance to one or more disease-modifying antirheumatic drugs.

58. The method of one of claims 1-57, wherein the patient has had an inadequate response or intolerance to methotrexate.

59. The method of any one of claims 1-58, wherein the patient has had an inadequate response or intolerance to an anti-malarial agent.

60. The method of any one of claims 1-59, wherein the patient has had an inadequate response or intolerance to an immunomodulator.

61. The method of any one of claims 1-60, wherein the patient has had an inadequate response or intolerance to a corticosteroid.

62. The method of any one of claims 1-61, wherein the patient has had an inadequate response or intolerance to prednisone (or prednisone equivalent), an antimalarial agent, azathioprine, mycophenolate, leflunomide, cyclosporine, tacrolimus, or methotrexate.

63. The method of any one of claims 1-62, wherein the patient has had an inadequate response or intolerance to belimumab.

64. The method of any one of claims 1-63, wherein the patient has had an inadequate response or intolerance to rituximab.

65. The method of any one of claims 1-64, wherein the patient has had an inadequate response or intolerance to anifrolumab.

66. The method of any one of claims 1-65, wherein the patient is concomitantly treated with an anti-malarial agent.

67. The method of one of claims 1-66, wherein the patient is concomitantly treated with an immunomodulator.

68. The method of any one of claims 1-67, wherein the patient is concomitantly treated with a corticosteroid.

69. The method of any one of claims 1-68, wherein the patient is concomitantly treated with prednisone (or prednisone equivalent), an antimalarial agent, azathioprine, mycophenolate, leflunomide, cyclosporine, tacrolimus, or methotrexate.

70. The method of any one of claims 1-69, wherein the patient is concomitantly treated with methotrexate.

71. The method of any one of claims 1-70, wherein the patient is concomitantly treated with azathioprine.

72. The method of any one of claims 1-71, wherein the patient is concomitantly treated with mycophenolate mofetil.

73. The method of any one of claims 1-72, wherein the patient is concomitantly treated with mycophenolate sodium.

74. The method of any one of claims 1-73, wherein the patient is concomitantly treated with hydroxychloroquine.

75. The method of any one of claims 1-74, wherein the patient is concomitantly treated with chloroquine.

76. The method of any one of claims 1-75, wherein the patient is concomitantly treated with quinacrine.

77. The method of any one of claims 1-76, wherein the patient is concomitantly treated with leflunomide.

78. The method of any one of claims 1-77, wherein the patient is concomitantly treated with cyclosporine.

79. The method of any one of claims 1-78, wherein the patient is concomitantly treated with tacrolimus.

80. The method of any one of claims 1-79, wherein the patient is concomitantly treated with prednisone or a prednisone equivalent.
